(19) United States
(12) Patent Application Publication
(10) Pub. No.: US 2024/0218068 A1
(43) Pub. Date: Jul. 4, 2024

(54) CANCER THERAPY USING CHECKPOINT INHIBITORS

(71) Applicant: TriSalus Life Sciences, Inc., Westminster, CO (US)

(72) Inventors: Steven C. Katz, East Greenwich, RI (US); Bryan F Cox, Muscatine, IA (US)

(73) Assignee: TriSalus Life Sciences, Inc., Westminster, CO (US)

(21) Appl. No.: 18/288,553

(22) PCT Filed: Apr. 29, 2022
(86) PCT No.: PCT/US2022/027093

§ 371 Date: Oct. 26, 2023

Related U.S. Application Data
(60) Provisional application No. 63/181,798, filed on Apr. 29, 2021.

(51) Int. Cl. C07K 16/28; A61P 35/00

(52) U.S. Cl. CPC C07K 16/2827; A61P 35/00; C07K 16/2818; C07K 2317/24; C07K 2317/76

(57) ABSTRACT

Embodiments of the present invention provide for methods of treating cancer and methods of delivering checkpoint inhibitors to solid tumors in the liver using a locoregional therapy through the vasculature. In one aspect, the present invention relates to a method of treating metastases of colorectal cancer of the liver comprising administering of checkpoint inhibitors to the liver. In another aspect, the present invention relates to a method of treating pancreatic cancer comprising administering checkpoint inhibitors to the pancreas.

### CROSS-REFERENCE TO RELATED APPLICATION(S)

This application claims the benefit of U.S. Provisional Patent Application No. 63/181,798, which was filed on Apr. 29, 2021, which is incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 16, 2020, is named A372-502_SL.txt and is 484 bytes in size.

### FIELD OF THE INVENTION

The present disclosure relates generally to methods of treating cancer and methods of delivering checkpoint inhibitors to solid tumors in the liver and/or pancreas using a locoregional therapy through the vasculature.

### BACKGROUND OF THE INVENTION

Cancer is a devastating disease that involves the unchecked growth of cells, which may result in the growth of solid tumors in a variety of organs such as the skin, liver, and pancreas. Tumors may first present in any number of organs or may be the result of metastasis or spread from other locations.

Checkpoint inhibitors (CPI) have revolutionized the treatment of certain solid tumors, including melanoma and non-small cell lung cancer. This class of therapy functions to inhibit checkpoint molecules within the solid tumor microenvironment (TME), one of the potent immune-evasive mechanisms utilized by tumors to evade immunity. Rather than directly attacking the tumor, CPI harness the power of the endogenous immune system by preventing the exploitation of the immune-evasive mechanisms tumors employ through the CTLA-4 and PD-1/PD-L1 pathways.

However, despite modest success with certain liver cancers (e.g., hepatocellular carcinoma and mismatch repair deficient stage IV adenocarcinomas), the impact of CPI therapy on liver tumors, in particular metastatic liver tumors, has been limited. In this regard, the current CPI therapies have resulted in insufficient hepatic activity and limited efficacy in the treatment of intrahepatic malignancies. This is particularly problematic for liver cancer patients, as immunosuppressive mechanisms in this organ are highly active. Further, the current CPI therapies have resulted in immune-related adverse events (irAEs). The severity of irAEs range from mild constitutional symptoms to severe organ failure and permanent debilitating effects such as pituitary insufficiency. Other examples of CPI-related irAEs include autoimmune-like toxicities such as colitis, dermatitis, and hepatitis. In this regard, CPIs have been associated with an alarmingly high frequency of irAEs, which is likely the result of high levels of systemic exposure during a systemic delivery (SD) of the CPI. In particular, during systemic delivery, the CPI binds, in a non-specific manner, to naturally occurring receptors present throughout the body that normally serve to regulate against self-antigen recognition, activation, and autoimmunity. As such, the emergence of irAEs may preclude continuation of an otherwise effective therapy, which limits the potential for durable control of advanced solid tumors.

Further, pancreatic cancer is the third leading cause of cancer deaths in the United States, responsible for an estimated 55,000 deaths in 2018. The 5-year survival rate of this type of cancer is only 7-8%, which is attributed to various factors including the advanced stage of the disease at which the initial diagnosis often occurs, the propensity of this type of cancer to metastasize, the resistance of the disease to chemotherapy and radiation therapy, and the complex microenvironment of pancreatic cancer tumors. Only 15-20% of patients are eligible at diagnosis for surgical resection of the primary tumor, as most patients are initially diagnosed with unresectable (metastatic or locally advanced) disease. The current standard of care for unresectable or metastatic pancreatic cancer is palliative systemic chemotherapy with either gemcitabine (Gem) monotherapy, gemcitabine/nab-paclitaxel, or folinic acid/fluorouracil/irinotecan/oxaliplatin (FOLFIRINOX). For patients with borderline resectable or locally advanced disease, combination regimens have been used to potentially convert some borderline resectable and even some locally advanced tumors to resectability. In addition, the relatively hypovascular immunosuppressive tumor microenvironment seen in most pancreatic adenocarcinomas makes targeted and comprehensive arterial delivery of chemotherapeutic agents challenging using conventional techniques.

Accordingly, there remains a need in the art for a more accurate, better-localized methods of delivering chemotherapy to treat solid tumors, such as colorectal cancer liver metastases (LM) and pancreatic cancer, that can address the limitations of current techniques.

### SUMMARY OF THE INVENTION

The present invention relates to methods of treating cancer and methods of delivering checkpoint inhibitors to solid tumors in the liver and/or pancreas using a locoregional therapy through the vasculature.

In one aspect, the present invention relates to a method of treating metastases of colorectal cancer of the liver comprising administering CPI through an intravascular device by hepatic arterial infusion (HAI). In another aspect, the present invention relates to a method of treating pancreatic cancer comprising administering CPI through an intravascular device by pancreatic retrograde venous infusion (PRVI).

In some embodiments, the CPI are administered through pressure-enabled drug delivery (PEDD).

In some embodiments, the CPI are administered through a pressure-enabled device.

In some embodiments, the CPI comprises a PD-1 antagonist.

In some embodiments, the PD-1 comprises one of nivolumab, pembrolizumab, and cemiplimab.

In some embodiments, the CPI comprises a PD-L1 antagonist.

In some embodiments, the PD-L1 antagonist is one of atezolizumab, avelumab, and durvalumab.

In some embodiments, the CPI is administered in combination with a toll-like receptor 9 agonist, such as SD-101.

These and other objects, features, and advantages of the exemplary embodiments of the present disclosure will become apparent upon reading the following detailed description of the exemplary embodiments of the present disclosure, when taken in conjunction with the appended paragraphs.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure.

FIG. 1 illustrates the structure of SD-101.

FIG. 2A illustrates a gating strategy of PD-L1 expression on MC38-CEA tumor cells according to an exemplary embodiment of the invention.

FIG. 2B illustrates a gating strategy of PD-L1 expression on G- and M-MDSCs according to an exemplary embodiment of the invention.

FIG. 3A illustrates a schematic representation of tumor development with MC38-CEA-luc and treatment timeline according to an exemplary embodiment of the invention.

FIG. 3B illustrates a graph depicting circulating levels of anti-PD-1 antibody in serum according to an exemplary embodiment of the invention.

FIG. 4 illustrates a liver function test according to an exemplary embodiment of the invention.

FIG. 5 illustrates graphs depicting effects of anti-PD-1 treatment on tumor growth according to an exemplary embodiment of the invention.

FIG. 6A illustrates a schematic representation of tumor development with MC38-CEA-luc and an exemplary TLR9 agonist treatment timeline according to an exemplary embodiment of the invention.

FIG. 6B illustrates a graph depicting effects of the exemplary TLR9 agonist treatment on tumor progression according to an exemplary embodiment of the invention.

FIG. 6C illustrates the effect of the exemplary TLR9 agonist on NFκB signaling according to an exemplary embodiment of the invention.

FIG. 7A illustrates a gating strategy for the exemplary TLR9 agonist according to an exemplary embodiment of the invention.

FIG. 7B illustrates the effect of the exemplary TLR9 agonist on the MDSC cell population according to exemplary embodiment of the invention.

FIG. 7C illustrates the effect of the exemplary TLR9 agonist on monocytic MDSCs (M-MDSC) according to exemplary embodiment of the invention.

FIG. 7D illustrates granulocytic MDSCs (G-MDSC) according to exemplary embodiment of the invention.

FIG. 7E illustrates another gating strategy for the exemplary TLR9 agonist according to exemplary embodiment of the invention.

FIG. 7F illustrates the effect of the exemplary TLR9 agonist on the M1-macrophage cell population according to an exemplary embodiment of the invention.

FIG. 7G illustrates the effect of the exemplary TLR9 agonist on the M2-macrophage cell population according to an exemplary embodiment of the invention.

FIG. 8A illustrates a secreted embryonic alkaline phosphatase (SEAP) assay used to evaluate exemplary TLR9 agonist-mediated NFκB activity according to an exemplary embodiment of the invention.

FIG. 8B illustrates the effect of chloroquine on the exemplary TLR9 agonist mediated NFκB activation and TNFα dependent activity according to an exemplary embodiment of the invention.

FIG. 9A illustrates a gating strategy for phenotypic analysis of MDSCs according to an exemplary embodiment of the invention.

FIG. 9B illustrates the effect of exemplary TLR9 agonists on huMDSC populations according to an exemplary embodiment of the invention.

FIG. 9C illustrates a Luminex analysis of the exemplary TLR9 agonists for (i) IL29, (ii) IFNα, (iii) IL6, and (iv) IL10, according to an exemplary embodiment of the invention.

FIG. 10A illustrates protein lysates obtained from patient biospecimens, and evaluated for TLR7 and TLR9 according to an exemplary embodiment of the invention.

FIG. 10B illustrates the expression of TLR9 in RNA isolated the patient biospecimens in FIG. 10A, according to an exemplary embodiment of the invention.

FIG. 10C illustrates the surface expression of TLR9 on MDSC cells according to an exemplary embodiment of the invention.

FIG. 10D illustrates the expression of TLR9 in human PMBC-derived MDSC cells according to an exemplary embodiment of the invention.

FIG. 11A illustrates a gating strategy to identify huMDSCs, its subtypes, and M1 macrophages according to an exemplary embodiment of the invention.

FIG. 11B illustrates the percentage of MDSCs for cells that were treated with the exemplary TLR9 agonist according to an exemplary embodiment of the invention.

FIG. 11C illustrates that ratio of M-MDSCs and G-MDSCs according to an exemplary embodiment of the invention.

FIG. 11D illustrates the M1 macrophage population according to an exemplary embodiment of the invention.

FIG. 11E illustrates the percentage of apoptotic MDSC cells according to an exemplary embodiment of the invention.

FIG. 11F illustrates the MDSC population after PBMCs were treated with the exemplary TLR9 agonist according to an exemplary embodiment of the invention.

FIG. 11G illustrates the phosphor STAT3 expression after PMBCs were treated with the exemplary TLR9 agonist according to an exemplary embodiment of the invention.

FIG. 12A illustrates a schematic representation of tumor development with MC38-CEA-luc and an exemplary TLR9 agonist and checkpoint inhibitor treatment timeline according to an exemplary embodiment of the invention.

FIG. 12B illustrates a graph depicting effects of the exemplary TLR9 agonist and checkpoint inhibitor treatment on tumor progression according to an exemplary embodiment of the invention.

FIG. 13 illustrates a densitometric analysis for the exemplary TLR9 agonist according to an exemplary embodiment of the invention.

FIG. 14 illustrates a Luminex analysis of the exemplary TLR9 agonists for (i) IL29, (ii) IFNα, (iii) IL6, and (iv) IL10, according to an exemplary embodiment of the invention.

FIG. 15 illustrates TLR9 expression in mouse L-MDSCs according to an exemplary embodiment of the invention.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to learn and understand the different described aspects of the invention. The description of embodiments should facilitate understanding of the invention to such an extent that other implementations, not specifically covered but within the knowledge of a person of skill in the art having read the description of embodiments, would be understood to be consistent with an application of the invention.

According to one embodiment, regional delivery of an anti-PD-1 agent for colorectal liver metastases improves therapeutic index and anti-tumor activity.

According to another embodiment, methods of the present invention may enhance intrahepatic effect while limiting extra-hepatic exposure.

According to yet another embodiment, methods of the present invention may provide enhanced tumor control and similar efficacy compared to higher doses of therapeutic agent administered via systemic delivery.

In another embodiment, methods of the present invention provide enhanced tumor control and similar efficacy compared to systemic delivery, wherein the dose administered by the present invention has over 10-fold lower concentration to the minimum effective systemic dose up to one week after treatment.

In some embodiments, the PD-1 antagonist and/or PD-L1 antagonist is administered in combination with another therapeutic, such as SD-101.

### Checkpoint Inhibitors

According to an embodiment, the CPI can include a Programmed Death 1 receptor (PD-1) antagonist. A PD-1 antagonist can be any chemical compound or biological molecule that blocks binding of Programmed Cell Death 1 Ligand 1 (PD-L1) expressed on a cancer cell to PD-1 expressed on an immune cell (T cell, B cell or NKT cell) and preferably also blocks binding of PD-L2 Programmed Cell Death 1 Ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1.

According to an embodiment, the PD-1 antagonist can include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

According to an embodiment, the PD-1 antagonist can include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule.

According to an embodiment, the PD-1 antagonist can inhibit the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain.

According to an embodiment, the PD-1 antagonist can be one of nivolumab, pembrolizumab, and cemiplimab.

According to another embodiment, the CPI can include a PD-L1 antagonist. In this regard, the PD-L1 antagonist can be one of atezolizumab, avelumab, and durvalumab.

### Toll-Like Receptor Agonists

Toll-like receptors are pattern recognition receptors that can detect microbial pathogen-associated molecular patterns (PAMPs). TLR stimulation, such as TLR9 stimulation, can not only provide broad innate immune stimulation, but can also specifically address the dominant drivers of immunosuppression in the liver. TLR1-10 are expressed in humans and recognize a diverse variety of microbial PAMPs. In this regard, TLR9 can respond to unmethylated CpG-DNA, including microbial DNA. CpG refers to the motif of a cytosine and guanine dinucleotide 1. TLR9 is constitutively expressed in B cells, plasmacytoid dendritic cells (pDCs), activated neutrophils, monocytes/macrophages, T cells, and MDSCs. TLR9 is also expressed in non-immune cells, including keratinocytes and gut, cervical, and respiratory epithelial cells. TLR9 can bind to its agonists within endosomes. Signaling may be carried out through MYD88/IkB/NfκB to induce pro-inflammatory cytokine gene expression. A parallel signaling pathway through IRF7 induces type 1 and 2 interferons (e.g. IFN-α, IFN-γ, etc.) which stimulate adaptive immune responses. Further, TLR9 agonists can induce cytokine and IFN production and functional maturation of antigen presenting dendritic cells.

According to an embodiment, a TLR9 agonist can reduce and reprogram MDSCs. MDSCs are key drivers of immunosuppression in the liver. MDSCs also drive expansion of other suppressor cell types such as T regulatory cells (Tregs), tumor-associated macrophages (TAMs), and cancer-associated fibroblasts (CAFs). MDSCs may downregulate immune cells and interfere with the effectiveness of immunotherapeutics. Further, high MDSC levels generally predict poor outcomes in cancer patients. In this regard, reducing, altering, or eliminating MDSCs is thought to improve the ability of the host's immune system to attack the cancer as well as the ability of the immunotherapy to induce more beneficial therapeutic responses. In an embodiment, TLR9 agonists may convert MDSCs into immunostimulatory M1 macrophages, convert immature dendritic cells to mature dendritic cells, and expand effector T cells to create a responsive tumor microenvironment to promote anti-tumor activity.

According to an embodiment, synthetic CpG-oligonucleotides (CPG-ONs) mimicking the immunostimulatory nature of microbial CpG-DNA can be developed for therapeutic use. According to an embodiment, the oligonucleotide is an oligodeoxynucleotide (ODN). There are a number of different CpG-ODN class types, e.g. Class A, Class B, Class C, Class P, and Class S, which share certain structural and functional features. In this regard, Class A type CPG-ODNs (or CPG-A ODNs) are associated with pDC maturation with little effect on B cells as well as the highest degree of IFNα induction; Class B type CPG-ODNs (or CPG-B ODNs) strongly induce B-cell proliferation, activate pDC and monocyte maturation, NK cell activation, and inflammatory cytokine production; and Class C type CPG-ODNs (or CPG-C ODNs) can induce B-cell proliferation and IFN-α production.

Further, according to an embodiment, CPG-C ODNs can be associated with the following attributes: (i) unmethylated dinucleotide CpG motifs, (ii) juxtaposed CpG motifs with flanking nucleotides (e.g. AACGTTCGAA), (iii) a complete phosphorothioate (PS) backbone that links the nucleotides (as opposed to the natural phosphodiester (PO) backbones found in bacterial DNA), and (iv) a self-complimentary, palindromic sequence (e.g. AACGTT). In this regard, CPG-C ODNs may bind themselves due to their palindromic nature, thereby producing double-stranded duplex or hairpin structures.

Further, according to an embodiment, the CPG-C ODNs can include one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In an embodiment, the CpG-C ODNs are 12 to 100 bases in length, preferably 12 to 50 bases in length, preferably 12 to 40 bases in length, or preferably 12-30 bases in length. In an embodiment, the CpG-C ODN is 30 bases in length. In an embodiment, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In an embodiment, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length.

In an embodiment, the at least one palindromic sequence is 8 to 97 bases in length, preferably 8 to 50 bases in length, or preferably 8 to 32 bases in length. In an embodiment, the at least one palindromic sequence is at least (lower limit) 8, 10, 12, 14, 16, 18, 20, 22, 24, 26, 28, or 30 bases in length. In an embodiment, the at least one palindromic sequence is at most (upper limit) 50, 48, 46, 44, 42, 40, 38, 36, 34, 32, 30, 28, 26, 24, 22, 20, 18, 16, 14, 12 or 10 bases in length.

In an embodiment, the CpG-C ODN can comprise the sequence of SEQ ID NO: 1.

According to an embodiment, the CpG-C ODN can comprise the SD-101. SD-101 is a 30-mer phosphorothioate oligodeoxynucleotide, having the following sequence: 5′-TCG AAC GTT CGA ACG TTC GAA CGT TCG AAT-3′ (SEQ ID NO: 1).

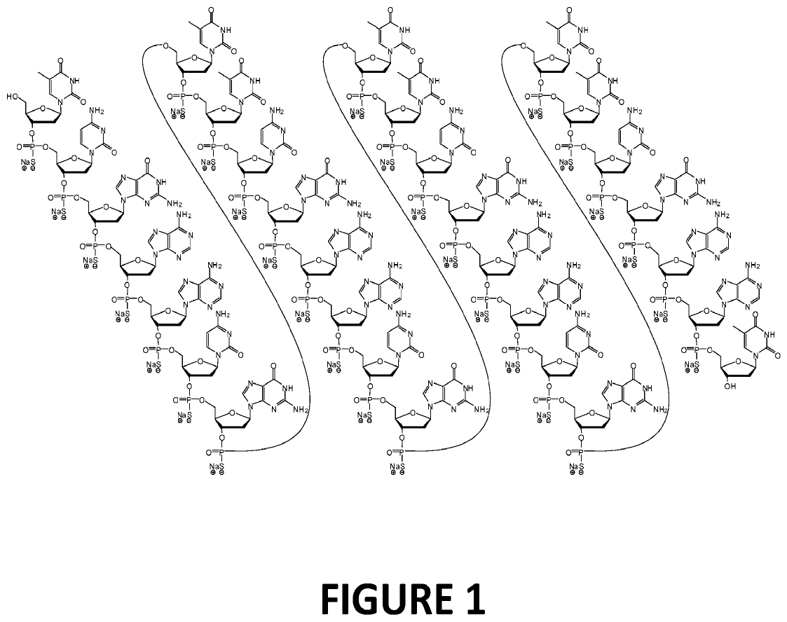

SD-101 drug substance is isolated as the sodium salt. The structure of SD-101 is illustrated in FIG. 1.

The molecular formula of SD-101 free acid is C₂₉₃H₃₆₉N₁₁₂O₁₄₉P₂₉S₂₉ and the molecular mass of the SD-101 free acid is 9672 Daltons. The molecular formula of SD-101 sodium salt is C₂₉₃H₃₄₀N₁₁₂O₁₄₉P₂₉S₂₉Na₂₉ and the molecular mass of the SD-101 sodium salt is 10,309 Daltons.

Further, according to an embodiment, the CPG-C ODN sequence can correspond to SEQ ID NO: 172 as described in U.S. Pat. No. 9,422,564, which is incorporated by reference herein in its entirety.

In an embodiment, the CpG-C ODN can comprise a sequence that has at least 75% homology to any of the foregoing, such as SEQ ID NO:1.

According to another embodiment the CPG-C ODN sequence can correspond to any one of the other sequences described in U.S. Pat. No. 9,422,564. Further, the CPG-C ODN sequence can also correspond to any of the sequences described in U.S. Pat. No. 8,372,413, which is also incorporated by reference herein in its entirety.

According to an embodiment, any of the CPG-C ODNs discussed herein may be present in their pharmaceutically acceptable salt forms. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In an embodiment, the CpG-C ODNs are in the ammonium, sodium, lithium, or potassium salt forms. In one preferred embodiment, the CpG-C ODNs are in the sodium salt form. The CpG-C ODN may be provided in a pharmaceutical solution comprising a pharmaceutically acceptable excipient. Alternatively, the CpG-C ODN may be provided as a lyophilized solid, which is subsequently reconstituted in sterile water, saline or a pharmaceutically acceptable buffer before administration. Pharmaceutically acceptable excipients of the present disclosure include for instance, solvents, bulking agents, buffering agents, tonicity adjusting agents, and preservatives. In an embodiment, the pharmaceutical compositions may comprise an excipient that functions as one or more of a solvent, a bulking agent, a buffering agent, and a tonicity adjusting agent (e.g. sodium chloride in saline may serve as both an aqueous vehicle and a tonicity adjusting agent). The pharmaceutical compositions of the present disclosure are suitable for parenteral and/or percutaneous administration.

In an embodiment, the pharmaceutical compositions comprise an aqueous vehicle as a solvent. Suitable vehicles include for instance sterile water, saline solution, phosphate buffered saline, and Ringer's solution. In an embodiment, the composition is isotonic.

The pharmaceutical compositions may comprise a bulking agent. Bulking agents are particularly useful when the pharmaceutical composition is to be lyophilized before administration. In an embodiment, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In an embodiment, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin, and mannitol.

The pharmaceutical compositions may comprise a preservative. Suitable preservatives include for instance antioxidants and antimicrobial agents. However, in an embodiment, the pharmaceutical composition is prepared under sterile conditions and is in a single use container, and thus does not necessitate inclusion of a preservative.

Table 1 describes the batch formula for SD-101 Drug Product—16 g/L:

TABLE 1

| | | | | Clinical Lot |
|---|---|---|---|---|
| | | | | DVXA05 |
| | | Concen- | Amount | Batch Size |
| Ingredient | Grade | tration | per batch | 2.224 L |
| SD-101 Drug | GMP | 1.6% | 16.00 g | 35.584 g¹ |
| Substance* | | | | |
| Sodium | USP/NF, EP | 1.02% | 1.02 g | 2.268 g |
| phosphate, | | | | |
| dibasic | | | | |
| anhydrous | | | | |
| Sodium | USP | 0.34% | 0.34 g | 0.747 g |
| phosphate, | | | | |
| monobasic | | | | |
| anhydrous | | | | |
| Sodium | USP/NF, EP | 7.31% | 7.31 g | 16.257 g |
| chloride | | | | |
| Sterile Water | USP/NF, EP | QS | QS | 2.224 L |
| for Injection | | | | (kg) (QS) |

¹Quantity based upon measured content in solution (to exclude moisture present in lyophilized powder)
*SD-101 Drug Substance in Table 1 reflects the totality of all oligonucleotide content, including SD-101.

In some embodiments, the unit dose strength may include from about 0.1 mg/mL to about 20 mg/mL. In one embodiment, the unit dose strength of SD-101 is 13.4 mg/mL.

In some embodiments, the amount of SD-101 administered is in the range of about 0.01-20 mg, or at least one of 0.5 mg, 2 mg, 4 mg, or 8 mg.

In some embodiments, SD-101 is administered in a solution in the range of 1-100 mL, or at least one of 10 mL, 25 mL, 30 mL, or 50 mL.

In some embodiments, an administered dose of SD-101 is in the range of 0.0001-20 mg/mL. In some embodiments, the administered dose of SD-101 is one of 0.01 mg/mL, 0.04 mg/mL, 0.08 mg/mL, or 0.16 mg/mL.

CpG-C ODNs may contain modifications. Suitable modifications can include but are not limited to, modifications of the 3′OH or 5′OH group, modifications of the nucleotide base, modifications of the sugar component, and modifications of the phosphate group. Modified bases may be included in the palindromic sequence as long as the modified base(s) maintains the same specificity for its natural complement through Watson-Crick base pairing (e.g. the palindromic portion of the CpG-C ODN remains self-complementary). Examples of modifications of the 5′OH group can include biotin, cyanine 5.5, the cyanine family of dyes, Alexa Fluor 660, the Alexa Fluor family of dyes, IRDye 700, IRDye 800, IRDye 800CW, and the IRDye family of dyes.

CpG-C ODNs may be linear, may be circular or include circular portions and/or a hairpin loop. CpG-C ODNs may be single stranded or double stranded. CpG-C ODNs may be DNA, RNA or a DNA/RNA hybrid.

CpG-C ODNs may contain naturally-occurring or modified, non-naturally occurring bases, and may contain modified sugar, phosphate, and/or termini. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. In an embodiment, CpG-C ODNs have only phosphorothioate linkages, only phosphodiester linkages, or a combination of phosphodiester and phosphorothioate linkages.

Sugar modifications known in the field, such as 2′-alkoxy-RNA analogs, 2′-amino-RNA analogs, 2′-fluoro-DNA, and 2′-alkoxy- or amino-RNA/DNA chimeras and others described herein, may also be made and combined with any phosphate modification. Examples of base modifications include but are not limited to addition of an electron-withdrawing moiety to C-5 and/or C-6 of a cytosine of the CpG-C ODN (e.g. 5-bromocytosine, 5-chlorocytosine, 5-fluorocytosine, 5-iodocytosine) and C-5 and/or C-6 of a uracil of the CpG-C ODN (e.g. 5-bromouracil, 5-chlorouracil, 5-fluorouracil, 5-iodouracil). As noted above, use of a base modification in a palindromic sequence of a CpG-C ODN should not interfere with the self-complementarity of the bases involved for Watson-Crick base pairing. However, outside of a palindromic sequence, modified bases may be used without this restriction. For instance, 2′-O-methyl-uridine and 2′-O-methyl-cytidine may be used outside of the palindromic sequence, whereas, 5-bromo-2′-deoxycytidine may be used both inside and outside the palindromic sequence. Other modified nucleotides, which may be employed both inside and outside of the palindromic sequence include 7-deaza-8-aza-dG, 2-amino-dA, and 2-thio-dT.

Duplex (i.e. double stranded) and hairpin forms of most ODNs are often in dynamic equilibrium, with the hairpin form generally favored at low oligonucleotide concentration and higher temperatures. Covalent interstrand or intrastrand cross-links increase duplex or hairpin stability, respectively, towards thermal-, ionic-, pH-, and concentration-induced conformational changes. Chemical cross-links can be used to lock the polynucleotide into either the duplex or the hairpin form for physicochemical and biological characterization. Cross-linked ODNs that are conformationally homogeneous and are “locked” in their most active form (either duplex or hairpin form) could potentially be more active than their uncross-linked counterparts. Accordingly, some CpG-C ODNs of the present disclosure can contain covalent interstrand and/or intrastrand cross-links.

The techniques for making polynucleotides and modified polynucleotides are known in the art. Naturally occurring DNA or RNA, containing phosphodiester linkages, may be generally synthesized by sequentially coupling the appropriate nucleoside phosphoramidite to the 5′-hydroxy group of the growing ODN attached to a solid support at the 3′-end, followed by oxidation of the intermediate phosphite triester to a phosphate triester. Using this method, once the desired polynucleotide sequence has been synthesized, the polynucleotide is removed from the support, the phosphate triester groups are deprotected to phosphate diesters and the nucleoside bases are deprotected using aqueous ammonia or other bases.

The CpG-C ODN may contain phosphate-modified oligonucleotides, some of which are known to stabilize the ODN. Accordingly, some embodiments include stabilized CpG-C ODNs. The phosphorous derivative (or modified phosphate group), which can be attached to the sugar or sugar analog moiety in the ODN, can be a monophosphate, diphosphate, triphosphate, alkylphosphonate, phosphorothioate, phosphorodithioate, phosphoramidate or the like.

CpG-C ODNs can comprise one or more ribonucleotides (containing ribose as the only or principal sugar component), deoxyribonucleotides (containing deoxyribose as the principal sugar component), modified sugars or sugar analogs. Thus, in addition to ribose and deoxyribose, the sugar moiety can be pentose, deoxypentose, hexose, deoxyhexose, glucose, arabinose, xylose, lyxose, and a sugar analog cyclopentyl group. The sugar can be in pyranosyl or in a furanosyl form. In the CpG-C oligonucleotide, the sugar moiety is preferably the furanoside of ribose, deoxyribose, arabinose or 2′-0-alkylribose, and the sugar can be attached to the respective heterocyclic bases in either anomeric configuration. The preparation of these sugars or sugar analogs and the respective nucleosides wherein such sugars or analogs are attached to a heterocyclic base (nucleic acid base) per se is known, and therefore need not be described here. Sugar modifications may also be made and combined with any phosphate modification in the preparation of a CpG-C ODN.

The heterocyclic bases, or nucleic acid bases, which are incorporated in the CpG-C ODN can be the naturally-occurring principal purine and pyrimidine bases, (namely uracil, thymine, cytosine, adenine and guanine, as mentioned above), as well as naturally-occurring and synthetic modifications of said principal bases. Thus, a CpG-C ODN may include one or more of inosine, 2′-deoxyuridine, and 2-amino-2′-deoxyadenosine.

According to another embodiment, the CPG-ODN is one of a Class A type CPG-ODNs (CPG-A ODNs), a Class B type CPG-ODNs (CPG-B ODNs), a Class P type CPG-ODNs (CPG-P ODN), and a Class S type CPG-ODNs (CPG-S ODN). In this regard, the CPG-A ODN can be CMP-001.

In another embodiment, the CPG-ODN can be tilsotolimod (IMO-2125).

### Devices to Achieve Locoregional Delivery

According to an embodiment, any of the above-described devices may comprise any device useful to achieve locoregional delivery to a tumor, including a catheter itself, or may comprise a catheter along with other components (e.g., filter valve, balloon, pressure sensor system, pump system, syringe, outer delivery catheter, implantable port, etc.) that may be used in combination with the catheter. In certain embodiments, the catheter is a microcatheter.

In some embodiments, the device may have one or more attributes that include, but are not limited to, self-centering capability that can provide homogeneous distribution of therapy in downstream branching network of vessels; anti-reflux capability that can block or inhibit the retrograde flow of the CPI or the TLR agonist (for example, with the use of a valve and filter, and/or balloon); a system to measure the pressure inside the vessel; and a means to modulate the pressure inside the vessel. In some embodiments, the system is designed to continuously monitor real-time pressure throughout the procedure.

In some embodiments, the device that may be used to perform the methods of the present invention is a device as disclosed in U.S. Pat. Nos. 8,500,775, 8,696,698, 8,696,699, 9,539,081, 9,808,332, 9,770,319, 9,968,740, U.S. Patent Publication No. 2018/0055620, U.S. Patent Publication No. 2018/019359, U.S. Patent Publication No. 2018/0250469, U.S. Patent Publication No. 2018/0263752, U.S. Patent Publication No. 2019/0111234, U.S. Patent Publication No. 2019/0298983, U.S. patent application Ser. No. 16/408,266, and U.S. patent application Ser. No. 16/431,547, which are all incorporated by reference herein in their entireties.

In some embodiments, the device is a device as disclosed in U.S. Pat. No. 9,770,319. In certain embodiments, the device may be a device known as the Surefire Infusion System.

In some embodiments, the device supports the measurement of intravascular pressure during use. In some embodiments, the device is a device as disclosed in U.S. patent application Ser. No. 16/431,547. In certain embodiments, the device may be a device known as the TriSalus Infusion System. In certain embodiments, the device may be a device known as the TriNav™ Infusion System. In certain embodiments, the device may be a device known as the SEAL Device.

In some embodiments, the CPI and/or TLR agonist may be administered through a device via PEDD. In some embodiments, the CPI and/or TLR agonist may be administered while monitoring the pressure in the vessel, which can be used to adjust and correct the positioning of the device at the infusion site and/or to adjust the rate of infusion. Pressure may be monitored by, for example, a pressure sensor system comprising one or more pressure sensors.

The rate of infusion may be adjusted to alter vascular pressure and/or flow, which may promote the penetration of the CPI into and/or binding of TLR agonist to the target tissue or tumor. In some embodiments, the rate of infusion may be adjusted and/or controlled using a syringe pump as part of the delivery system. In some embodiments, the rate of infusion may be adjusted and/or controlled using a pump system. In some embodiments, the rate of infusion may be about 0.1 cc/min to about 40 cc/min, or about 0.1 cc/min to about 30 cc/min, or about 0.5 cc/min to about 25 cc/min, or about 0.5 cc/min to about 20 cc/min, or about 1 cc/min to about 15 cc/min, or about 1 cc/min to about 10 cc/min, or about 1 cc/min to about 8 cc/min, or about 1 cc/min to about 5 cc/min. In some embodiments, the rate of infusion is about 1-5 cc/sec.

### Methods Comprising Administration to the Liver

In an embodiment, the methods of the present invention include methods of treating a solid tumor in the liver, such as a tumor that is the metastasis of colorectal cancer, said method comprising administering CPI to a patient in need thereof, wherein the CPI is administered through a device by HAI to such solid tumor in the liver. HAI refers to the infusion of a treatment into the hepatic artery of the liver. According to an embodiment, the CPI are introduced through the percutaneous introduction of a device into the branches of a hepatic artery or portal vein, such as a catheter and/or a device that facilitates pressure-enabled delivery. In some embodiments, the catheter and/or the device comprises a one-way valve that responds dynamically to local pressure and/or flow changes. According to an embodiment, the CPI comprises a PD-1 antagonist or PD-L1 antagonist. In one embodiment, the patient is a human patient.

According to another embodiment, the tumor is unresectable.

In another embodiment, the methods of the present invention include methods of treating a solid tumor in the liver, such as a tumor that is the metastasis of colorectal cancer, said method comprising administering CPI in combination with a TLR agonist to a patient in need thereof, wherein the CPI and the TLR agonist are administered through a device by HAI to such solid tumor in the liver. HAI refers to the infusion of a treatment into the hepatic artery of the liver. According to an embodiment, the CPI and TLR agonist are introduced through the percutaneous introduction of a device into the branches of a hepatic artery or portal vein, such as a catheter and/or a device that facilitates pressure-enabled delivery. In some embodiments, the catheter and/or the device comprises a one-way valve that responds dynamically to local pressure and/or flow changes. According to an embodiment, the CPI comprises a PD-1 antagonist or PD-L1 antagonist. According to an embodiment, the TLR agonist is a TLR9 agonist. In some embodiments, the TLR9 agonist is SD-101. In some embodiments, the CPI is administered either concurrently, before, or after the administration of the TLR agonist. In some embodiments, the CPI is administered systemically. In one embodiment, the patient is a human patient.

In one embodiment, the above methods of administration to the liver are intended to result in the penetration of the CPI and/or the TLR agonist throughout the solid tumor, throughout the entire organ, or substantially throughout the entire tumor. In an embodiment, such methods enhance perfusion of the CPI and/or the TLR agonist to a patient in need thereof, including by overcoming interstitial fluid pressure and solid stress of the tumor. In another embodiment, perfusion throughout an entire organ or portion thereof, may provide benefits for the treatment of the disease by thoroughly exposing the tumor to therapeutic agent. In an embodiment, such methods are better able to afford delivery of the CPI and/or the TLR agonist to areas of the tumor that have poor access to systemic circulation. In another embodiment, such methods deliver higher concentrations of the CPI and/or the TLR agonist into such a tumor with less CPI and/or TLR agonist delivered to nontarget tissues compared to conventional systemic delivery via a peripheral vein. Nontarget tissues are tissues directly perfused by the arterial network in immediate connection with the infusion device. In one embodiment, such methods result in the reduction in size, reduction in growth rate, or shrinkage or elimination of the solid tumor.

The methods of the present invention may also include mapping the vessels leading to the right, left, and caudate lobes of the liver, or the various segments or sectors, prior to performing a HAI, and when necessary, occluding vessels which do not lead to the liver or as otherwise necessary. In some embodiments, prior to infusion, patients can undergo a mapping angiogram, e.g., via a common femoral artery approach.

Methods for mapping vessels in the body and delivery of therapeutics are well known to the ordinarily skilled artisan. Occlusion may be achieved, for example, through the use of microcoil embolization, which allows the practitioner to block off-target arteries or vessels, thereby optimizing delivery of the modified cells to the liver. Microcoil embolization can be performed as needed, such as prior to administering the first dose of CPI to facilitate optimal infusion of the CPI. In another embodiment, a sterile sponge (e.g., GELFOAM) can be used. In this regard, the sterile sponge can be cut and pushed into the catheter. In another embodiment, the sterile sponge can be provided as granules.

### Methods Comprising Administration to the Pancreas

In an embodiment, the methods of the present invention include methods of treating pancreatic cancer, said method comprising administering CPI to a patient in need thereof, wherein the CPI is administered through a device by PRVI to a solid tumor in the pancreas. PRVI refers to the infusion of a treatment to a solid tumor in the pancreas via a branch or branches of the pancreatic venous drainage system. According to an embodiment, the CPI are introduced through the percutaneous transhepatic introduction of a device into the branch(es) of the pancreatic venous drainage system, such as a catheter and/or a device that facilitates pressure-enabled delivery. According to an embodiment, the CPI comprises a PD-1 antagonist or a PD-L1 antagonist. In one embodiment, the patient is a human patient.

In an embodiment, delivery of the treatment by PRVI can be a more effective route of providing the CPI to pancreatic tumors. In particular, in contrast to systemic intravenous and locoregional intra-arterial therapies, PRVI can be used to provide treatment to the tumor without relying on the arterial supply to the tumor, and, therefore may be a more effective means of delivering the CPI and treating pancreatic cancer. For example, with PRVI, the CPI can be delivered to the tumor via a sub-selective, catheter-directed approach utilizing the draining veins of the targeted pancreatic tumor. For example, the CPI can be delivered to the tumor in a branch or branches of the pancreatic venous drainage system. In this regard, a digital subtraction angiography with computed tomography (CT) can be used to catheterize the veins draining the pancreatic tumor with a delivery device (e.g., catheter and/or a device that facilitates pressure-enabled delivery) in order to deliver the CPI in a retrograde fashion.

In an embodiment, the methods of the present invention include methods of treating pancreatic cancer, said method comprising administering CPI to a patient in need thereof, wherein the CPI is administered through a device by infusion through the pancreatic arterial system to a solid tumor in the pancreas. According to an embodiment, the CPI is introduced through the percutaneous introduction of a device into the pancreatic arterial system, such as a catheter and/or a device that facilitates pressure-enabled delivery. For example, the pancreatic arterial system can be accessed by means of the splenic artery, the gastroduodenal artery, or the inferior pancreatic duodenal artery. In this regard, the head can be accessed through the gastroduodenal artery to the anterior and posterior pancreatic duodenal arteries, while the body and tail can be accessed from the splenic artery to the dorsal pancreatic artery, the great pancreatic artery, or the caudal pancreatic artery. From these vessels, smaller feeding vessels can be selected as required for the treatment of the target tissue. According to an embodiment, the CPI is a PD-1 antagonist or PD-L1 antagonist. In one embodiment, the patient is a human patient.

In another embodiment, the methods of the present invention include methods of treating pancreatic cancer, said method comprising administering CPI in combination with a TLR agonist to a patient in need thereof, wherein the CPI and TLR agonist are administered through a device by infusion through the pancreatic arterial system to a solid tumor in the pancreas. According to an embodiment, the CPI and TLR agonist are introduced through the percutaneous introduction of a device into the pancreatic arterial system, such as a catheter and/or a device that facilitates pressure-enabled delivery. For example, the pancreatic arterial system can be accessed by means of the splenic artery, the gastroduodenal artery, or the inferior pancreatic duodenal artery. In this regard, the head can be accessed through the gastroduodenal artery to the anterior and posterior pancreatic duodenal arteries, while the body and tail can be accessed from the splenic artery to the dorsal pancreatic artery, the great pancreatic artery, or the caudal pancreatic artery. From these vessels, smaller feeding vessels can be selected as required for the treatment of the target tissue. According to an embodiment, the CPI is a PD-1 antagonist or PD-L1 antagonist. According to an embodiment, the TLR agonist is a TLR9 agonist. In some embodiments, the TLR9 agonist is SD-101. In some embodiments, the CPI is administered either concurrently, before, or after the administration of the TLR agonist. In some embodiments, the CPI is administered systemically. In one embodiment, the patient is a human patient.

The pancreatic cancer can comprise a solid tumor in the pancreas, such as an exorcine tumor, such as a pancreatic adenocarcinoma. Examples include, but are not limited to, ductal adenocarcinoma (including pancreatic ductal adenocarcinoma and locally advanced pancreatic ductal adenocarcinoma) and acinar adenocarcinoma. In an embodiment, the tumor is unresectable or resection is not a reasonable undertaking due to the presence of advanced disease. Further, in an embodiment, the tumor is a metastatic pancreatic adenocarcinoma.

In one embodiment, the above methods of administration to the pancreas are intended to result in the penetration of the CPI and/or TLR agonist throughout the solid tumor, throughout the entire organ, or substantially throughout the entire tumor. In an embodiment, such methods enhance perfusion of the CPI and/or TLR agonist to a patient in need thereof, including by overcoming interstitial fluid pressure and solid stress of the tumor. In another embodiment, perfusion throughout an entire organ or portion thereof, may provide benefits for the treatment of the disease by thoroughly exposing the tumor to therapeutic agent. In an embodiment, such methods are better able to afford delivery of the CPI and/or TLR agonist to areas of the tumor that have poor access to systemic circulation. In another embodiment, such methods deliver higher concentrations of the CPI and/or TLR agonist into such a tumor with less CPI and/or TLR agonist delivered to nontarget tissues compared to conventional systemic delivery via a peripheral vein. Nontarget tissues are tissues directly perfused by the arterial network in immediate connection with the infusion device. In one embodiment, such methods result in the reduction in size, reduction in growth rate, or shrinkage or elimination of the solid tumor.

### Administered Doses for Liver and Pancreas

In some embodiments, doses of the CPI may be about 0.01 mg/kg, about 0.03 mg/kg, about 0.05 mg/kg, about 0.1 mg/kg, about 0.3 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, or about 8 mg/kg.

In some embodiments, doses of the CPI may be between about 0.01 mg/kg and about 20 mg/kg, about 0.01 mg/kg and about 10 mg/kg, between about 0.01 mg/kg and about 8 mg/kg, and between about 0.01 mg/kg and about 4 mg/kg. In some embodiments, doses the CPI may be between about 2 mg/kg and about 10 mg/kg, between about 2 mg/kg and about 8 mg/kg, and between about 2 mg/kg and about 4 mg/kg. In some embodiments, doses of the CPI may be less than about 10 mg/kg, less than about 8 mg/kg, less than about 4 mg/kg, or less than about 2 mg/kg. Such doses may be administered daily, weekly, every other week, every third week, every fourth week, etc., or whatever is considered to be clinical best practice. In one embodiment, doses of the CPI are incrementally increased, such as through administration of about 0.3 mg/kg, followed by about 1 mg/kg, then followed by 3.0 mg/kg, and then followed by about 5.0 mg/kg.

In some embodiments, doses of a TLR9 agonist, such as SD-101 may be about 0.01 mg, about 0.03 mg, about 0.05 mg, about 0.1 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, or about 8 mg. In some embodiments, SD-101 is administered at doses of 12 mg, 16 mg, and 20 mg. Administration of a milligram amount of SD-101 (e.g. about 2 mg) describes administering about 2 mg of the composition illustrated in FIG. 1. For example, such an amount of SD-101 (e.g. about a 2 mg amount) may also exist within a composition that contains material in addition to such amount of SD-101, such as other related and unrelated compounds. Equivalent molar amounts of other pharmaceutically acceptable salts are also contemplated.

In some embodiments, doses of a TLR9 agonist, such as SD-101, may be between about 0.01 mg and about 20 mg, about 0.01 mg and about 10 mg, between about 0.01 mg and about 8 mg, and between about 0.01 mg and about 4 mg. In some embodiments, doses of a TLR9 agonist, such as SD-101 may be between about 2 mg and about 10 mg, between about 2 mg and about 8 mg, and between about 2 mg and about 4 mg. In some embodiments, doses of a TLR9 agonist, such as SD-101 may be less than about 10 mg, less than about 8 mg, less than about 4 mg, or less than about 2 mg. Such doses may be administered daily, weekly, or every other week. In one embodiment, doses of SD-101 are incrementally increased, such as through administration of about 2 mg, followed by about 4 mg, and then followed by about 8 mg.

In one or more embodiments, a solution of SD-101 may be administered to a subject via HAI using a TriNav® device to perform PEDD. In some such embodiments, vascular access may be achieved using the femoral artery, radial artery, or brachial artery approach. Hemangiomata, shunting vessels, or other vascular lesions in the liver that may interfere with therapeutic delivery may be embolized at the discretion of the treating interventional radiology specialist. In one or more embodiments, the SD-101 can be prepared and delivered in a 50 mL syringe (therapeutic dose) and a 100-mL vial containing the volume necessary for the therapeutic flush (10 mL), both at the therapeutic concentration. The pressure modulating device can then be advanced into the target vessels.

In one or more embodiments, the 50 mL solution of SD-101 can be allocated by per segment or sector of the liver. In one or more embodiments, the 50-mL therapeutic dose of SD-101 can be allocated as follows: 3×10 mL infusions into target blood vessels in the right hepatic lobe and 2×10 mL infusions into target blood vessels in the left hepatic lobe. Further, the distribution of the 10-mL aliquots may be adjusted based on the location of measurable disease and target vessel diameter. In one or more embodiments, the SD-101 infusion can be expected to last approximately 10-60 minutes. For example, in some embodiments, the infusion time can be approximately 25 minutes. Further, in another embodiment, the overall interventional procedure can last between 30-80 minutes. This involves all the handling time between infusions in different locations. In some embodiments, the 50 mL solution of SD-101 can include one of 0.5 mg, 2 mg, 4 mg, or 8 mg of SD-101. In this regard, the infused dose of SD-101 can be one of 0.01 mg/mL, 0.04 mg/mL, 0.08 mg/mL, or 0.16 mg/mL.

According to another embodiment, the SD-101 can be prepared and delivered in a 25 mL solution. In some embodiments, the 25 mL solution of SD-101 can include one of 0.5 mg, 2 mg, 4 mg, or 8 mg of SD-101. In this regard, the infused dose of SD-101 can be one of 0.02 mg/mL, 0.08 mg/mL, 0.16 mg/mL, or 0.32 mg/mL.

According to another embodiment, the SD-101 can be prepared and delivered in a 10 mL solution. In some embodiments, the 10 mL solution of SD-101 can include one of 0.5 mg, 2 mg, 4 mg, or 8 mg of SD-101. In this regard, the infused dose of SD-101 can be one of 0.05 mg/mL, 0.2 mg/mL, 0.4 mg/mL, or 0.8 mg/mL.

In some embodiments, the methods of the present invention result in the treatment of target lesions. In this embodiment, the methods of the present invention may result in a complete response, comprising the disappearance of all target lesions. In some embodiments, the methods of the present invention may result in a partial response, comprising at least a 30% decrease in the sum of the longest diameter of target lesions, taking as reference the baseline sum longest diameter. In some embodiments, the methods of the present invention may result in stable disease of target lesions, comprising neither sufficient shrinkage to qualify for partial response nor sufficient increase to qualify for progressive disease, taking as reference the smallest sum longest diameter since the treatment started. In such an embodiment, progressive disease is characterized by at least a 20% increase in the sum of the longest diameter of target lesions, taking as reference the smallest sum longest diameter recorded since the treatment started or the appearance of one or more new lesions. The sum must demonstrate an absolute increase of 5 mm.

In another embodiment, the methods of the present invention result in the treatment of nontarget lesions. Nontarget lesions are lesions not directly perfused by the arterial network in immediate communication with the infusion system. In this embodiment, the methods of the present invention may result in a complete response, comprising the disappearance of all nontarget lesions. In some embodiments, the methods of the present invention result in persistence of one or more nontarget lesion(s), while not resulting in a complete response or progressive disease. In such an embodiment, progressive disease is characterized by unequivocal progression of existing nontarget lesions, and/or the appearance of one or more new lesions.

In some embodiments, the methods of the present invention result in an increased duration of overall response. In some embodiments, the duration of overall response is measured from the time measurement criteria are met for complete response or partial response (whichever is first recorded) until the first date that recurrent or progressive disease is objectively documented (taking as reference for progressive disease the smallest measurements recorded since the treatment started). The duration of overall complete response may be measured from the time measurement criteria are first met for complete response until the first date that progressive disease is objectively documented. In some embodiments, the duration of stable disease is measured from the start of the treatment until the criteria for progression are met, taking as reference the smallest measurements recorded since the treatment started, including the baseline measurements.

In yet other embodiments, the methods of the present invention result in improved overall survival rates. For example, overall survival may be calculated from the date of enrollment to the time of death. Patients who are still alive prior to the data cutoff for final efficacy analysis, or who dropout prior to study end, will be censored at the day they were last known to be alive.

In other embodiments, the methods of the prevent invention result in progression-free survival. For instance, progression-free survival may be calculated from the date of enrollment to the time of CT scan documenting relapse (or other unambiguous indicator of disease development), or date of death, whichever occurs first. Patients who have no documented relapse and are still alive prior to the data cutoff for final efficacy analysis, or who drop out prior to study end, will be censored at the date of the last radiological evidence documenting absence of relapse.

According to another embodiment, the methods of the present invention result in a reduction of tumor burden. In some embodiments, the tumor burden is reduced by about 10%, by about 20%, by about 30%, by about 40%, by about 50%, by about 60%, by about 70%, by about 80%, by about 90%, or by about 100%.

According to another embodiment, the methods of the present invention results in a reduction of tumor progression or stabilization of tumor growth. In some embodiments, tumor progression is reduced by about 10%, by about 20%, by about 30%, by about 40%, by about 50%, by about 60%, by about 70%, by about 80%, by about 90%, or by about 100%.

According to another embodiment, the methods of the present invention result in the reprogramming of the liver MDSC compartment to enable immune control of the liver cancer and/or improves responsiveness to systemic anti-PD-1 therapy through elimination of MDSC. In some embodiments, the methods of the present invention are superior in controlling MDSC. In some embodiments, the methods of the present invention reduce the frequency of MDSC cells, monocytic MDSC (M-MDSC) cells, granulocytic MDSC (G-MDSC) cells, or human MDSC. According to another embodiment, the methods of the present invention enhance M1 macrophages. According to yet another embodiment, the methods of the present invention decrease M2 macrophages.

In another embodiment, the methods of the present invention increase NFκB activation. In yet an additional embodiment, the methods of present invention increase IL-6. In another embodiment, the methods of the present invention increase IL-10. In yet an additional embodiment, the methods of present invention increase IL-29. In another embodiment, the methods of the present invention increase IFNα. As a further embodiment, the methods of the present invention decrease STAT3 phosphorylation.

The present invention will be further illustrated and/or demonstrated in the following Examples, which is given for illustration/demonstration purposes only and is not intended to limit the invention in anyway.

### Example 1

In the present example, it was hypothesized that regional delivery (RD) of CPI can improve anti-tumor activity in the liver and minimize systemic exposure.

### Materials and Methods

### Murine Colorectal Cancer Liver Metastases Model

Six to ten-week old C57BL/6J male mice were anesthetized using aerosolized isoflurane and 2.5×10⁶ MC38-CEA cells with a luciferase reporter protein (MC38-CEA-luc) were delivered via splenic injection to generate colorectal cancer liver metastases (CRCLM) followed by splenectomy to confine tumor growth to the liver. Post-operatively, buprenorphine (0.05-0.1 mg/kg) or buprenorphine SR (0.5-1 mg/kg) was injected subcutaneously for analgesia and treated with SQ 0.9% saline.

### Bioluminescence Monitoring and Quantification

Mice were anesthetized as above and 100 μL of XenoLight D-Luciferin was delivered via intraperitoneal (IP) injection followed by gentle peritoneal massage to ensure adequate distribution. The mice were placed individually in the IVIS machine and imaged under auto-exposure with a maximum exposure time of 60 seconds and an F/Stop of 1.2 with XFOV Lens in place. Each mouse was imaged three days after tumor inoculation to establish baseline tumor burden prior to treatment and on each post-treatment day (PTD) subsequently. Tumor bioluminescence (TB) was quantified as total flux (protons/s) using LivingImage 4.7.2 software with values that were normalized to the baseline (DO) bioluminescence value (photons/s). Bioluminescence <1.0×10⁵ photons/s at DO was considered as background and thus mice needed a TB of >1.0×10⁵ photons/s for inclusion in the study.

### CPI Delivery

After baseline IVIS imaging, tumor-bearing mice were treated with 0.3 mg/kg, 1.0 mg/kg, 3.0 mg/kg, or 5.0 mg/kg of a Rat IgG2a Isotype anti-mouse PD-1 antibody (e.g., RMP1-14) via the portal vein (PV) for RD or tail vein (TV) for SD or phosphate buffered saline (PBS) via PV served as vehicle control. Doses were selected based on the standard weight-based dosing used in human trials. For PV delivery, a sterile catheter composed of polyurethane tubing (0.017 in ID×0.037 in OD) attached to a 30G access needle was attached to a 25G blunt tipped needle and 10 mL syringe for infusion. The syringe was placed in an automated pressure injector and target volumes were set accordingly.

Once the injection catheter was prepared, exploratory laparotomy was performed on tumor-bearing mice anesthetized as above. With the liver retracted cranially, the 30G needle was used to cannulate the PV and advanced until just proximal to the bifurcation of the right and left hepatic branches. Therapy was administered and the access needle was removed while maintaining manual pressure over the insertion site for hemostasis. Once achieved, the organs were replaced in their anatomic positions and the fascia closed with 4-0 Vicryl followed by skin clips for the skin. The mice in the TV cohort were anesthetized as mentioned above and placed in a restraint chamber with tails submerged in warmed water to dilate the lateral tail veins. Once sufficient dilation was achieved, a 30G ½″ Needle was attached to a 1 ml syringe and therapy was delivered. Analgesia and fluid replacement were provided post-operatively and all mice placed in a warmed chamber.

### Cell Isolation

Liver non-parenchymal cell (L-NPC) isolation was performed with several modifications. Mice were euthanized via terminal cardiac puncture and immediately following, the CRCLM-bearing liver was explanted and a portion of the tissue was placed directly into a gentleMACS™ C tube with RPMI 1640 and enzymes from the Tissue Dissociation Kit for mechanical disruption with the gentleMACS™ dissociator. Samples were incubated at 37° C. for 40 minutes prior to a second round of dissociation and the resulting cell suspension was washed through a 70 μm MACS SmartStrainer with RPMI 1640. Hepatocytes were separated out via low-speed centrifugation followed by density gradient separation using 40% Optiprep and Gey's Balanced Salt Solution. The remaining cells were ACK lysed with lysis buffer, incubated with 1 μg of anti-FcγR III/II mAb2.4G2, and isolated for CD45+ cells with CD45 immuno-magnetic beads to obtain L-NPC without liver sinusoidal endothelial cells (LSEC) containing 30% CD11b⁺ L-MDSC on average (quantified per approximately 35,000 cells on average). Isolated cells were stained immediately for flow cytometry or cryopreserved for later studies.

### Flow Cytometry and Antibodies

Isolated L-MDSC and tumor cells were stained with antibodies specific for murine CD11b, Ly6C, Ly6G, PD-L1 and human CD66 to assess MDSC and tumor phenotypes with regards to expression of PD-L1. These antibodies were conjugated to combinations of FITC, BV421, PE-Cy7, and APC (CD11b-FITC, Ly6C-BV421, Ly6G-PECy7, CD66-FITC, and PD-L1-APC) based on the study of interest and fluorophore combination. Results were analyzed with FlowJo 10.6.1 and gating performed using unstained cells and single stain controls.

### Serum Studies

Euthanasia was performed via terminal cardiac puncture with blood collected in 1.5 mL Eppendorf tubes. The blood was allowed to coagulate for 4-6 hours at 4° C. Serum was separated from the blood by spinning in a microcentrifuge for 10 minutes at 2,000 rcf and the serum was transferred to a new 1.5 mL Eppendorf tube. The serum was diluted with deionized water total volume of 200 μL and sent for complete metabolic panel and bilirubin analysis. The remaining serum was used for enzyme-linked immunosorbent assay (ELISA) or cryopreserved.

Serum harvested from the mice as above was plated on a sandwich ELISA kit designed to detect rat IgG2a proteins against a standard curve according to the manufacturers protocol. Colorimetric changes were detected using a SPECTROstar Nano absorbance reader and sample absorbance was interpolated against the standard curve.

### Western Blots

Tumors were washed twice with ice-cold PBS and lysed with RIPA buffer supplemented with protease inhibitor cocktail, as described previously. Protein quantification was performed using Bradford protein assay using BSA as the standard. Lysates were denatured using Laemmli sample buffer with freshly added β-mercaptoethanol. The immunoblots were analyzed and quantified using ImageJ software. Antibodies to PD-1 (e.g., D7D5W), PD-L1 (e.g., B7-H1), cleaved caspase 9 (D3Z2G), Ki-67 (SolA15) and GAPDH (D4C₆R) were used at a 1:500 dilution.

### Statistics

Statistical analysis was performed using Prism 8. Data are displayed as mean±standard error of mean (SEM) with corresponding values of n. Statistical significance was calculated using student's t-test and ANOVA. Values with p≤0.05 were determined to be significant. Group-based Grubb's test was performed on bioluminescence to mathematically identify outliers which were excluded from the study. Using both criteria, n=1-2 animals were excluded from analysis in each of the eight groups uniformly.

### Results

### Liver Metastases Promote Immunosuppression in the Tumor Microenvironment Via the PD-L1/PD-1 Axis

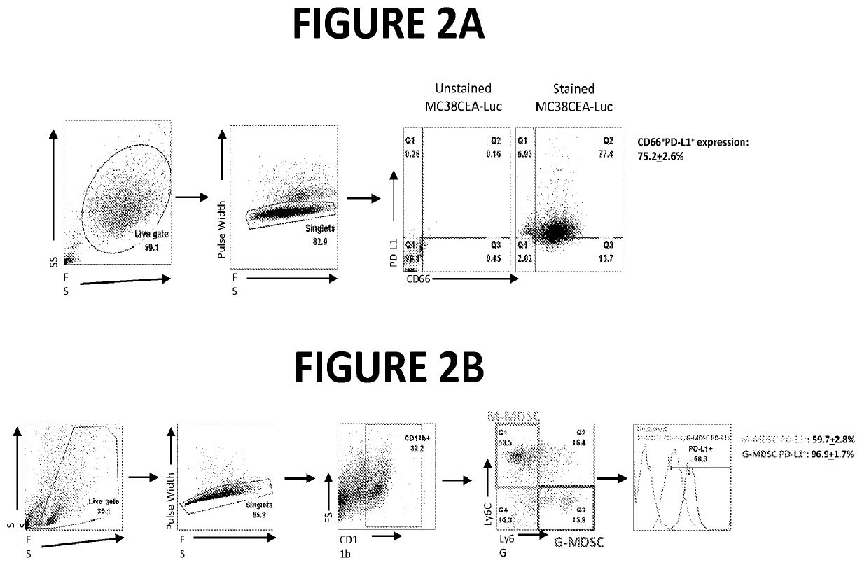

It was hypothesized that CRCLM cells would mediate immunosuppression through the PD-1/PD-L1 axis as well as creating a tumor microenvironment (TME) that further exacerbated this. To confirm expression levels of PDL-1 in the liver TME, tumor and suppressor cell expression of this protein were examined. After 48 hours in culture, 75.2+2.6% of MC38-CEA-luc cells expressed PD-L1 (see FIG. 2A). FIG. 2A illustrates a gating strategy of PD-L1 expression on MC38-CEA tumor cells. As depicted in the figure, after doublet cell exclusion and co-staining with CD66 (CEA), PD-L1 antibodies showed high expression of PD-L1 on tumor cells. Further, Evaluation of expression was performed in biological replicates, where n=3.

Further, high expression of 96.9+1.7% of PD-L1 was observed in granulocytic MDSC (G-MDSC) and 59.7+2.8% was observed in monocytic MDSC (M-MDSC) (FIG. 2B). FIG. 2B illustrates a gating strategy of PD-L1 expression on G- and M-MDSCs according to an exemplary embodiment of the invention. As depicted in the figure, after doublet cell exclusion, G-MDSC was identified as CD11b⁺Ly6G^{hi}Ly6C^{lo} and M-MDSC was identified as CD11b⁺Ly6G^{lo}Ly6C^{hi} phenotypes, respectively. One box denotes M-MDSC and another box denotes G-MDSC. Further, the evaluation of expression was performed in biological replicates, where n=4.

### Anti-PD-1 Antibody Effective as In Vivo Checkpoint Inhibitor Therapy Against Tumors

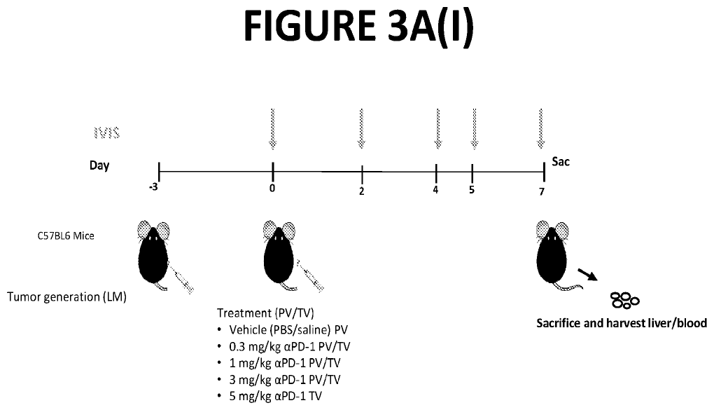

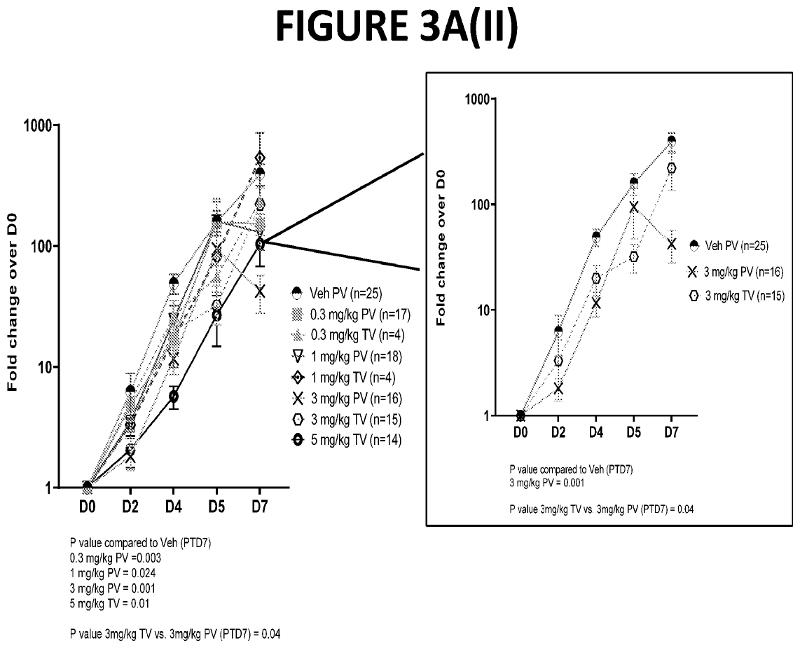

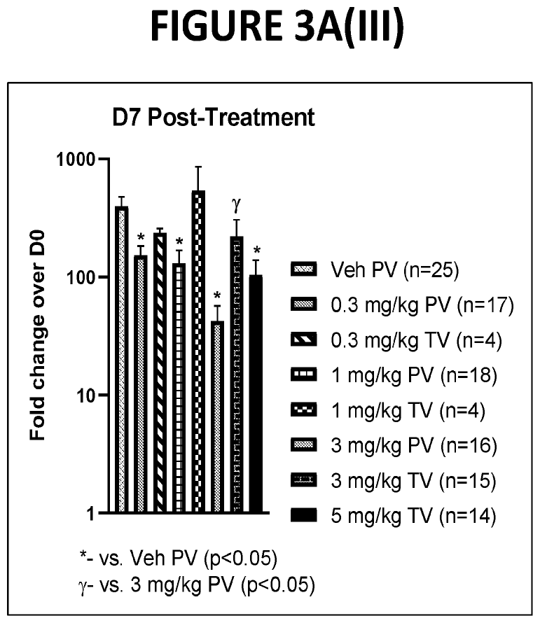

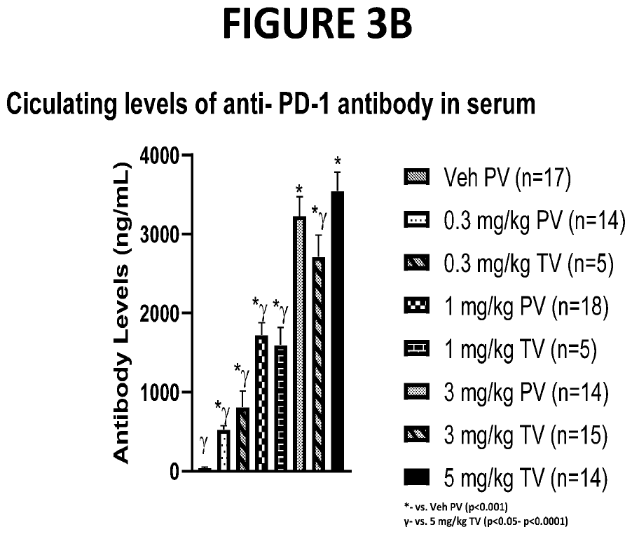

To investigate the effect in TME of anti-PD-1 treatment, mice were challenged with intra-splenic MC38-CEA-luc to generate LM followed by treatment on day 3 with varying concentrations (0.3 mg/kg-5 mg/kg) of anti-PD-1 treatment delivered via TV or PV, as shown in FIG. 3A. Specifically, the mice separated into eight treatment groups and treated according to the schema depicted with vehicle control (Veh) via portal vein (PV) or 0.3 mg/kg, 1 mg/kg, 3 mg/kg tail vein (TV) or PV and 5 mg/kg TV. Number of mice for each group is shown in the graphs. Bioluminescence (arrows) was measured using IVIS imaging on post-treatment day (PTD)0 (baseline), PTD2, PTD4, PTD5 and PTD7 and represented as fold over PTD0 in log scale. Right graph shows the inset of Veh PV, 3 mg/kg PV and 3 mg/kg TV and PTD7 bioluminescence comparison of different doses and routes of administration. Results are shown as mean±SEM. The in vivo results revealed route specific response in TB at the standard dose of 3 mg/kg on PTD7 via PV as compared to 3 mg/kg via TV (p=0.04) and compared to vehicle control (p=0.001) (see FIG. 3A). Significant differences were seen in TB for all escalating doses delivered via PV compared to vehicle control and only in 5 mg/kg TV as compared to vehicle control (PTD7 p<0.05). The minimal effective dose at PTD7 was 5 mg/kg (p=0.01) via TV and 0.3 mg/kg (p=0.02) via PV compared to vehicle control indicating a lower dose requirement via RD to achieve similar anti-tumor activity as observed with SD. No significant difference between delivery routes, PV or TV was observed for any time point for any of the lower doses (0.3 mg/kg, 1 mg/kg).

Reduction in Systemic Exposure with Low Dose Regional Delivery

Using an ELISA assay, the serum of treated mice was assessed for levels of circulating anti-PD-1 antibody and all doses were detectable compared to the vehicle control cohort (p<0.001 for all comparisons, see FIG. 3B). Specifically, circulating levels of anti-PD-1 antibody were assessed on PTD7 to determine levels of systemic exposure in the serum via a sandwich ELISA against Rat IgG2a proteins. With regards to route of delivery, large differences were found between the amounts of detected antibody. When comparing similar doses, there were no significant differences found between 3 mg/kg PV vs. 3 mg/kg TV (3233.10 vs. 2714.09 ng/mL, p=0.17). However, the lower RD doses at 0.3 mg/kg PV and 1 mg/kg PV both resulted in significantly less detected antibody in circulation compared to all higher doses regardless of route of delivery (0.3 mg/kg PV p<0.001 for all comparisons, 1.0 mg/kg PV p<0.01-p<0.001). There was a 6.7-fold reduction in comparing 0.3 mg/kg PV to the minimum efficacious systemic dose of 5 mg/kg TV (p<0.001). Additionally, the 3 mg/kg TV cohort revealed lower amounts of antibody compared to 5 mg/kg TV (p=0.03) indicating that an increase in circulating anti-PD-1 antibody does not necessarily correlate to improved response (see FIG. 3A).

Equivalent Hepatic Toxicity Comparing Delivery Methods with Similar Doses

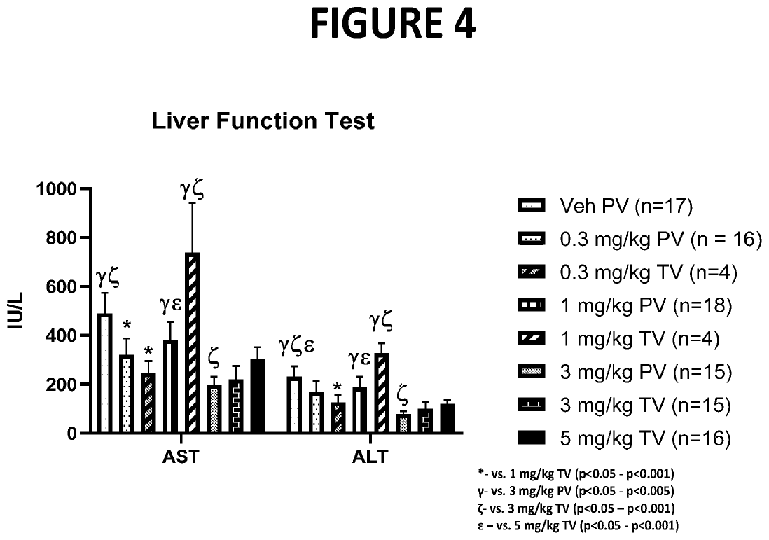

Serum was assessed for liver toxicities with analysis of liver function tests (LFT) including aspartate transaminase (AST) and alanine transaminase (ALT) of each treatment group. Significant differences were seen in LFTs when performing ANOVA analysis across all of the groups (AST p=0.005, ALT p=0.004, see FIG. 4). This is likely influenced by the elevations in AST and ALT seen in the vehicle control and 1 mg/kg TV cohorts, which when examined with the bioluminescence data is suggestive of hepatic damage secondary to tumor burden rather than toxicity from treatment. The equivalent AST and ALT levels when comparing higher dose SD and the RD doses also supports this conclusion while indicating that RD techniques do not appreciably result in additional liver tissue damage. Importantly, there was no significant increase in AST or ALT due to the administered doses via PV or TV as compared to vehicle control, indicating no anti-PD-1 related liver toxicity.

### PD-1 Inhibition Promotes Apoptotic Signaling in Tumor

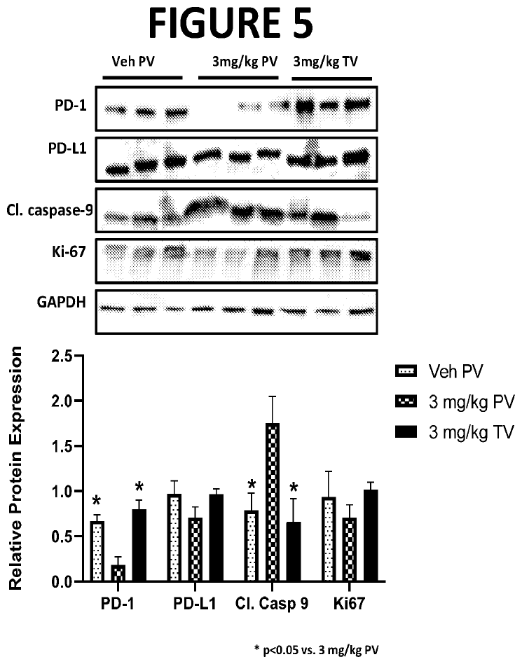

To evaluate the apoptosis/proliferation of tumor cells due to PD-1 inhibition, LM tumor lysates from PTD3 were isolated from three mice from 3 mg/kg TV, 3 mg/kg PV and vehicle control groups, respectively. Specifically, the tumor lysates from vehicle control, 3 mg/kg PV and 3 mg/kg TV were analyzed by western blot with antibodies against PD-1, PD-L1, cleaved caspase-9, Ki-67. The immunoblots were reprobed with antibody against GAPDH as a loading control. Triplicate samples were loaded (n=3 mice/group) and the signals were quantified using densitometric analysis and normalized with GAPDH protein expression. Results are shown as mean+SEM. In this regard, western blot analysis of PD-1 showed significantly low protein expression in 3 mg/kg PV as compared to vehicle control (p<0.05), which confirmed increased inhibition of PD-1 in PV as compared to TV or vehicle control with no change in PD-L1 expression in the tumors (FIG. 5). Further, there was significantly increased apoptosis as seen by enhanced levels of cleaved caspase 9 in 3 mg/kg PV tumor lysates as compared to vehicle control and 3 mg/kg TV groups (p<0.05). There was also a decreasing trend, though not significant, in Ki67 (proliferation) expression in 3 mg/kg PV as compared to 3 mg/kg TV and vehicle control.

### Description of Data

The experimental model produced CRCLMs that enhanced control when RD was employed with similar efficacy to higher dose SD. Further, RD results in similar efficacy even with over 10-fold lower concentration to the minimum effective systemic dose up to one week after treatment. Variation of biological effect is dependent on which ligand, PD-L1 or PD-L2 binds to PD-1. One model shows reverse roles of PD-L1 and PD-L2 signaling in activation of natural killer T cells. Inhibition of PD-L2 leads to enhanced T helper 2 cell activity, while PD-L1 binding to CD80 has been shown to inhibit T-cell responses. Blocking PD-1 facilitates inhibition of signaling via both PD-L1 and PD-L2 axis.

Further, RD strategies here avoid undesirable effects associated with SD (e.g., higher levels of systemic exposure, higher risk of irAEs, etc.) by directing therapy to the target site while maintaining therapeutic efficacy. Also, 3 mg/kg PV showed a decrease in PD-1 expression as compared to 3 mg/kg TV and vehicle control on PTD3 probably due to anti-PD-1 antibody causing local neutralization of PD-1 in TME, which further caused an increase in apoptosis of tumors that correlated with the significant decrease in TB in 3 mg/kg PV treated mice.

### Results of Study

Using a murine model of CRCLM, it was confirmed that there were high levels of PD-L1 expression on the tumor cells and liver myeloid-derived suppressor cells (L-MDSC). In vivo, the minimal efficacious dose via tail vein (TV) SD at post treatment day 7 (PTD7) was 5 mg/kg compared to vehicle control (p=0.01), and 0.3 mg/kg, (p=0.02) following RD via the portal vein (PV) compared to vehicle control. 6.7-fold lower circulating CPI antibody levels in the serum was detected using the 0.3 mg/kg PV treatment compared to the 5 mg/kg TV cohort (p<0.001) without increased liver toxicity. Also, 3 mg/kg PV treatment resulted in improved tumor killing compared to 5 mg/kg TV (p<0.05).

In summary, it has been demonstrated that RD of anti-PD-1 antibody can overcome the SD related auto-immune toxicities and provide comparable anti-tumor efficacy with over 10 fold lower concentration as compared to the minimum effective systemic dose. In other words, RD of an anti-PD-1 CPI therapy for CRCLM may improve the therapeutic index by reducing the total dose required and limiting the systemic exposure.

### Example 2

In the present example, it was hypothesized that the regional intra-vascular infusion of class C TLR9 agonists can reprogram the L-MDSC compartment to create a more immune-responsive TME.

### Materials/Subjects and Methods

### Mice, In Vivo Model, and Treatment

C57BL/6J, aged 7-10 weeks male mice, were obtained and housed under pathogen-free conditions. LM was generated by injecting 2.5×10⁶ MC38-CEA Luc cells via the spleen, followed by splenectomy. MC38-CEA was tested for mycoplasma prior to use. In vivo bioluminescence imaging was performed by using IVIS Lumina II Imaging System to monitor tumor burden on D0, D1, and D2. Mice were randomized into treatment groups so that animals in each group had a similar tumor burden. After seven days (D0), mice were treated with 1, 3, 10, or 30 μg/mouse of ODN2395 via PV or 30 μg/mouse of ODN2395 via TV. PV infusions were done with the Pressure Enabled Drug Delivery™ (PEDD™) infusion model for enhanced flow and delivery pressure. Mice treated with PBS via PV were used as control. Mice were sacrificed on D2, and livers were harvested. Liver non-parenchymal cells (NPCs) were isolated, and CD45⁺ cells were purified using immuno-magnetic beads as described previously. Isolated CD45⁺NPCs were then evaluated for MDSCs and macrophages (M1 and M2). To evaluate combinatorial effect of CPI and ODN2395, LM-bearing mice received 250 μg/mouse of anti-mouse PD-1 antibody (Clone: RMP1-14, Bio X Cell) intraperitoneally (IP) on D0, D3 and D10 and 30 μg/mouse ODN2395 via PV on DO. Number of mice used for each experiment was determined using G Power software and experimental replicates (biological and/or technical) are mentioned in respective figure legends. Mice were excluded from study if tumors were not generated or were sub-optimal (<10⁶ photons/s) as determined by in vivo bioluminescence imaging.

### Protein Analyses

Harvested mouse liver lysates were used in western blotting (WB) as described previously. Samples were washed twice with ice-cold PBS and lysed with RIPA buffer in the presence of protease inhibitor cocktail. Samples were homogenized using porcelain beads as per manufacturer's protocol. Sonicated samples were then centrifuged at 10,000 rpm for 10 minutes at 4° C. and the supernatant was collected. Protein quantification was performed using the Bradford protein assay using BSA as the standard. Lysates were denatured using Laemmli sample buffer with β-mercaptoethanol and denatured by heating the samples at 95° C. for 5 min. Electrophoresis was performed using Mini Protean TGX 4-15% gels and transferred on Trans-Blot Turbo PVDF membrane.

Cell supernatants obtained from in vitro experiments were tested for IL6, IL10, IL29, and IFNα using Procartaplex Luminex kit and measured by Magpix. For immunofluorescence (IF), huPBMC were grown in chamber slides. After fixing, cells were blocked and incubated with primary antibodies (1:100) at 37° C. for 1 hour. Secondary antibodies (1:250) conjugated with appropriate fluorophore were incubated at room temperature for 1 hour. Secondary antibody-only incubated samples served as negative controls for the procedure. Prolong-DAPI was used for nuclear staining. All images were captured using a Zeiss LSM 700 confocal laser-scanning microscope at 63× magnification. For flow cytometry (FC), 2.5×10⁵ cells incubated with antibodies for 30 minutes at room temperature, stained with BioLegend Zombie NIR (human cells only) for 30 minutes at room temperature, fixed with Cytofix and ran on a CytoFLEX LX flow cytometer. Compensation beads were used to set compensation and isotype controls were used to set gates. Flow cytometry data was analyzed by using CytExpert software.

### SEAP Assay

For TLR9-dependent NFκB reporter assay, HEK293-Blue cells were used. Cells were generated by co-transfecting the murine TLR9 gene and an inducible SEAP reporter gene into HEK293 cells. The SEAP gene was placed under the control of the interferon-beta (IFNβ) minimal promoter fused to five NFκB and activator protein-1 (AP-1) binding sites. Stimulation with a TLR9 ligand activates NFκB and AP-1, which induce the production of SEAP and are measured by a plate reader at 650 nm. Cells were treated with ODN2395 and SD-101 at increasing doses (0.004-10 μM) for 21 hours. Further, ODN5328(C) was used as a negative sequence control for ODN2395. In this regard, the sequence control contains GpC dinucleotides instead of CpG present in ODN2395.

### Results

### Regional Delivery of TLR9 Agonist Via PV Inhibits the Progression of LM

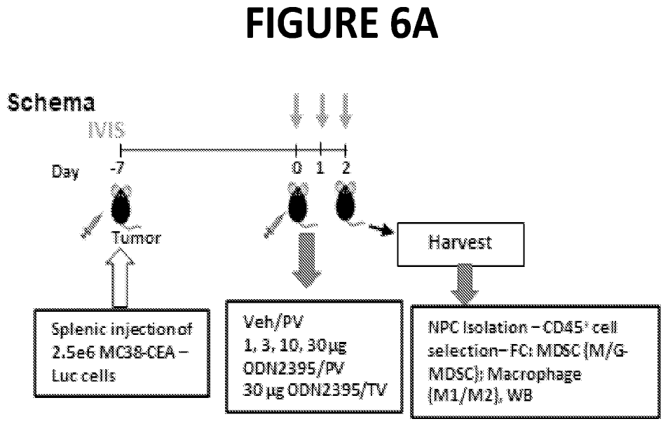

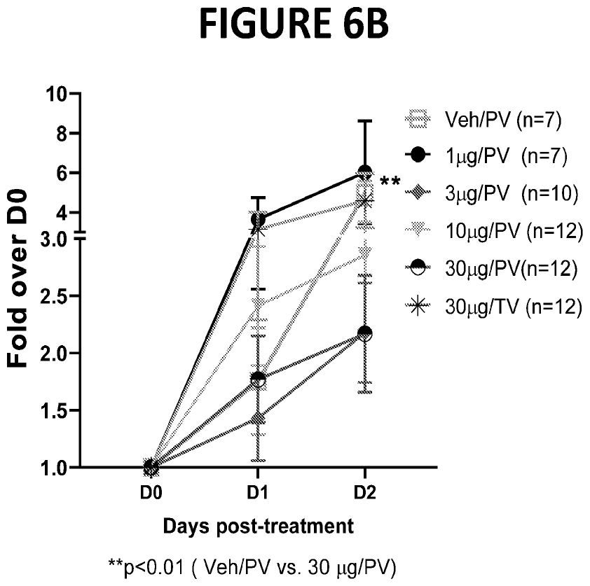

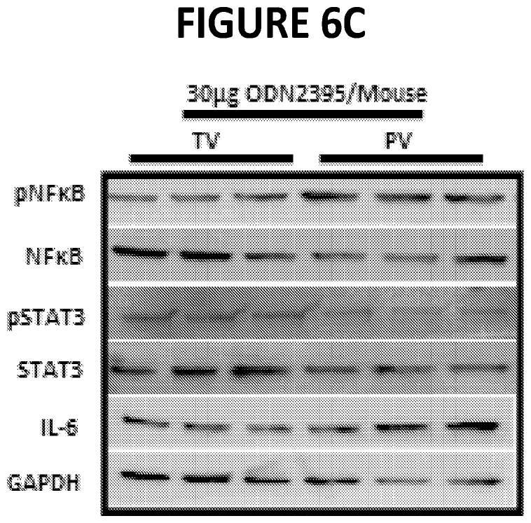

To evaluate single agent activity of a class C TLR9 agonist delivered by regional intravascular infusion using the PEDD™ murine model to enhance flow and pressure, LM-bearing mice were treated with 1, 3, 10, or 30 μg of class C ODN2395 via PV or TV (30 μg) per the schema (FIG. 6A). Bioluminescence was measured at baseline, 24- and 48-hours post-ODN2395 administration to quantify LM burden. It was found that 30 μg ODN2395 via PV had significantly improved tumor killing compared to vehicle control at D2 (bioluminescence fold change=1.08 vs. 2.80; p<0.01) (FIG. 6B), whereas TV infusion did not improve tumor control. There was a non-significant trend towards decreased tumor burden with 3 μg (D1; p=0.10; D2; p=0.14) and 30 μg ODN2395 (D1; p=0.16; D2; p=0.11) via PV as compared to 30 μg TV. In this regard, fold change of the tumor burden was calculated based on DO baseline bioluminescence. Multiple t test was performed to determine the significant difference (*p<0.05).

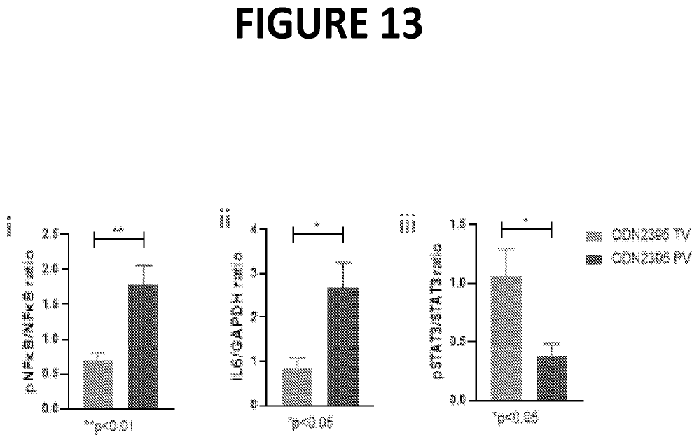

Class C ODNs activate both NFκB and IFNα pathways. It was hypothesized that a TLR9 agonist delivered regionally would result in enhanced NFκB activation as compared to systemic administration. In this regard, LM tissues (whole lysates) were harvested from n=6 mice/group (representative of n=3 shown) and evaluated for pNFκB (p65^{S536}), pSTAT3^{Y705}, total NFκB, STAT3, and IL6 by WB. GAPDH was used as a housekeeping protein control. After the LMs were harvested and the WBs were performed, it was found that mice that received 30 μg ODN2395 via PV had enhanced pNFκB/NFκB (1.77 vs. 0.68; p<0.01) ratio along with increased IL6 (2.7 vs. 0.8; p<0.05) expression and reduced pSTAT3/STAT3 (1.066 vs. 0.3865; p<0.05) ratio as compared to mice treated via TV (FIG. 6C, FIG. 13). FIG. 13 illustrates the ratio of (i) phospho/total NFκB, (ii) IL6/GAPDH and (iii) phospho/total STAT3 after a densitometric analysis was performed. Results are shown as mean±SEM. Students' t-test was performed (*p<0.05, **p<0.01; n=6).

### Class C TLR9 Agonists Delivered Via PV Alter the Immunosuppressive Phenotype of Myeloid Cells and Promote M1 Macrophage Polarization

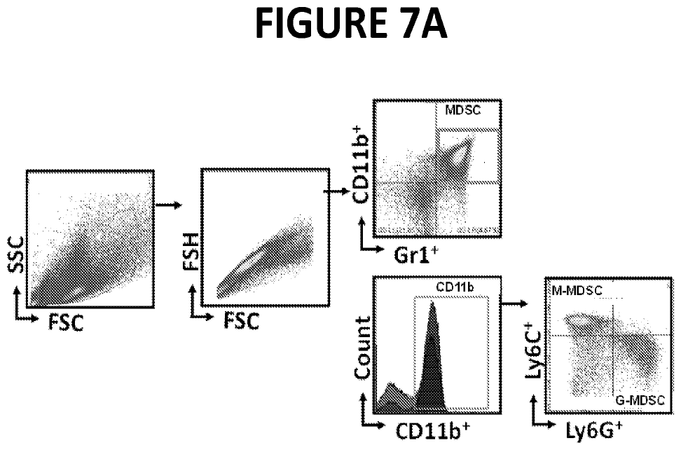

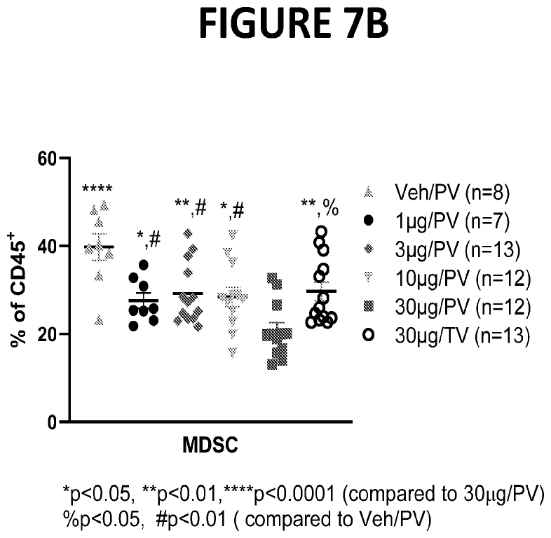

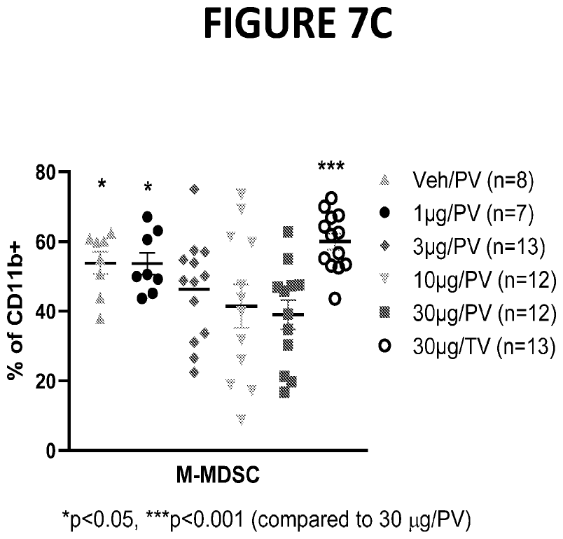

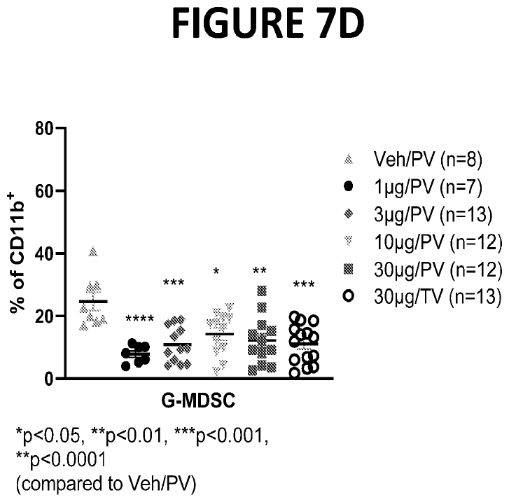

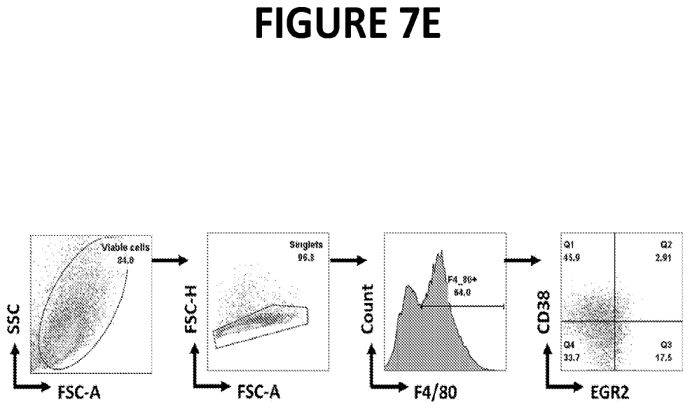

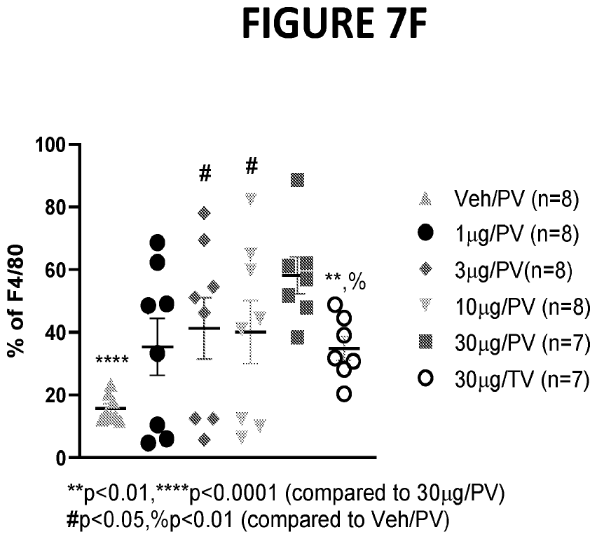

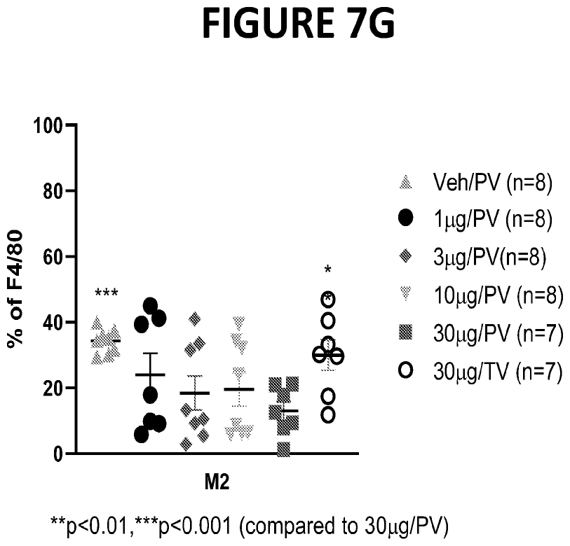

To investigate the effect of class C TLR9 agonists on immunosuppressive LM-MDSC expansion, bulk hepatic NPCs from LM-bearing mice were enriched with CD45⁺ cells and the frequency of MDSCs was measured (FIG. 7A, which shows a gating strategy to analyze CD45⁺ cells isolated from the NPCs). Mice that received 30 μg ODN2395 via PV had a significantly reduced LM-MDSC population as compared to vehicle (Veh) control (20.75% vs. 39.78%; p<0.0001) (FIG. 7B, which shows the measured MDSC cell population (CD11b⁺Gr1⁺)). Treating mice with 30 μg ODN2395 via PV was superior in reducing total MDSCs (20.75% vs. 29.70%; p<0.01) and M-MDSCs (38.98% vs. 60.03%; p<0.001) (FIG. 7C, which shows the measured M-MDSC (CD11b⁺Ly6C^{+/hi}Ly6G^{/lo})) as compared to the same dose via TV. In addition, low PV doses (10 μg and 3 μg) reduced M-MDSC relative to TV in a non-significant manner. The liver G-MDSC population was affected similarly by all doses and routes (FIG. 7D, which shows the measured G-MDSC (CD11b⁺Ly6C^{-/lo}Ly6G^{+/hi})).

M2 (F4/80⁺CD38⁻Egr2⁺) macrophages, like MDSCs, are immunosuppressive, while M1 (F4/80⁺CD38⁺Egr2⁻) macrophages mediate anti-tumor immune responses. As determined by FC (FIG. 7E, a gating strategy of the phenotypic analysis of CD45⁺ derived macrophages that were isolated from the LM), mice treated with ODN2395 had a significantly increased M1 macrophage cell population (FIG. 7F, which illustrates the determined M1-macrophage cell population (F4/80⁺CD38⁺Egr2⁻)) compared to the control group (p<0.05), irrespective of the route of delivery except for 1 μg/PV group. However, liver M1 macrophage polarization was significantly increased when the class C TLR9 agonist was delivered via PV (58.20% 30 μg PV vs. 34.82%; p<0.01 30 μg TV) in 30 μg ODN2395/PV compared to 30 μg/TV group. The M2 population was significantly reduced in mice treated with 30 μg ODN2395/PV compared to vehicle (12.99% vs. 29.96%; p<0.01) and 30 μg/TV (12.99% vs. 34.30%; p<0.0001) treated mice, as shown in FIG. 7G (determined M2-macrophage cell population (F4/80⁺CD38⁻Egr2⁺)). Accordingly, 30 μg ODN2395, when delivered via PV, significantly increased M1/M2 ratio as compared to the TV group (p<0.05). In this regard, each animal data is represented by a scattered plot and presented as mean±SEM from at least three different experiments. Students' t-test was performed for group-wise comparison and are described in each graph.

### ODN2395 and SD-101 Activate NFκB Signaling Via TLR9 Activation in a Non-Linear Manner

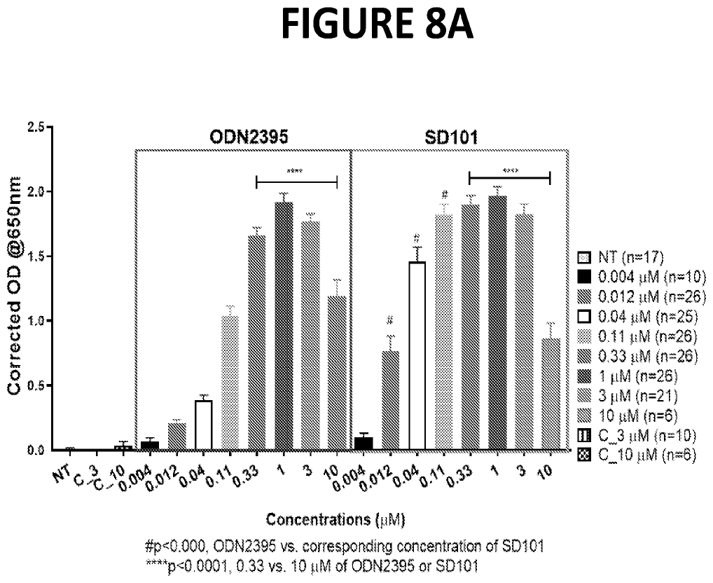

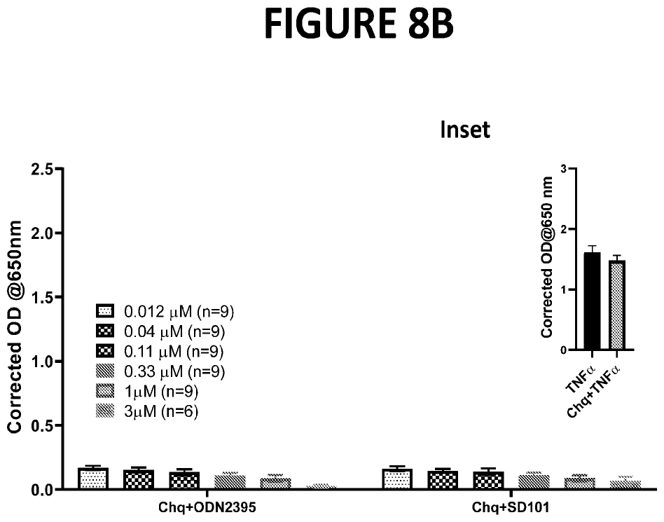

It was demonstrated that regional intravascular delivery of a class C TLR9 agonist enhanced NFκB phosphorylation. The potency of ODN2395 was then compared with SD-101. Specifically, a reporter-based assay in which TLR9-expressing HEK293-Blue cells were treated with ODN2395 and SD-101 at increasing doses (0.004-10 μM) for 21 hours. As a negative control, no-treatment (NT) and sequence control ODN5328 at 3 (C_3) and 10 (C_10) μM were used. The SEAP was determined by measuring the absorbance at 650 nm after addition of substrate. In this regard, similar non-linear dose-dependent responses were observed for both ODN2395 and SD-101 with respect to TLR9 signaling activity (FIG. 8A). The negative sequence control ODN5328 at 3 and 10 μM, as well as non-treated cells, did not produce appreciable SEAP (FIG. 8A). The requirement for TLR9 activity in activating NFκB was determined by pretreating cells with 1 μg/mL chloroquine (Chq) for 45 minutes, an inhibitor of endosomal maturation, prior to exposure to ODN2395 or SD-101 at increasing concentrations (0.012-3 μM) for 21 hours, with absorbance measured at 650 nm. In this regard, All the experiments were performed at least three times with 2-3 replicates, and mean±SEM was plotted in the graph. FIG. 8B shows that Chq completely inhibited NFκB activation by ODN2395 and SD-101-treated cells (0.012-3 μM). However, cells pretreated with Chq followed by canonical NFκB activation by tumor necrosis factor-alpha (TNFα; 20 ng/ml) stimulation did not affect SEAP production (FIG. 8B inset).

Class C TLR9 Agonists Reduced Human Peripheral MDSC In Vitro while Enhancing PBMC NFκB- and IFNα-Dependent Cytokines

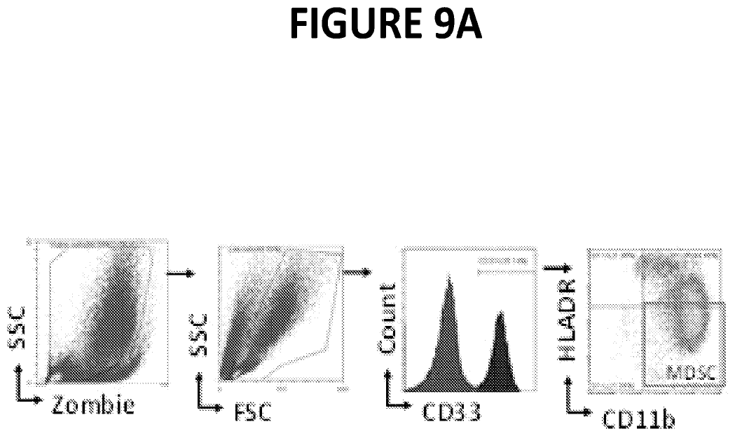

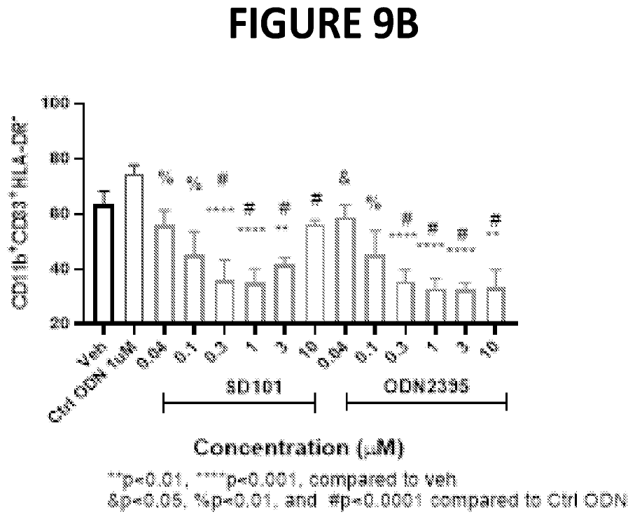

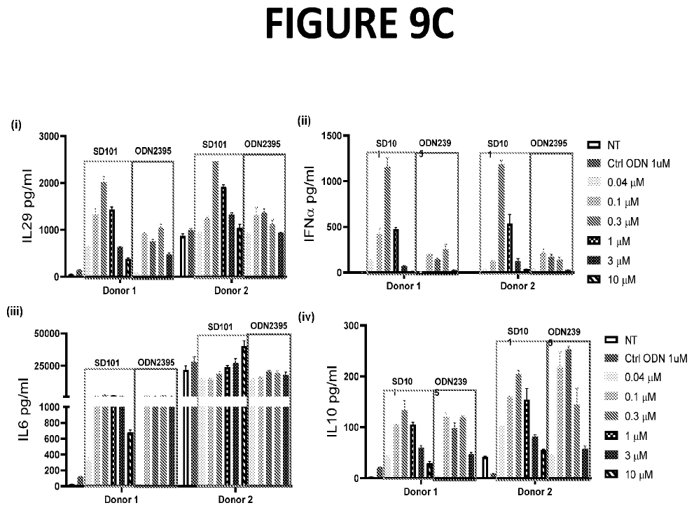

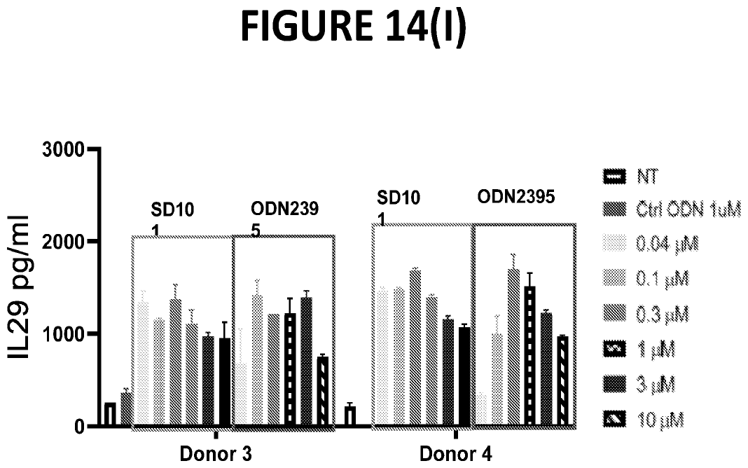

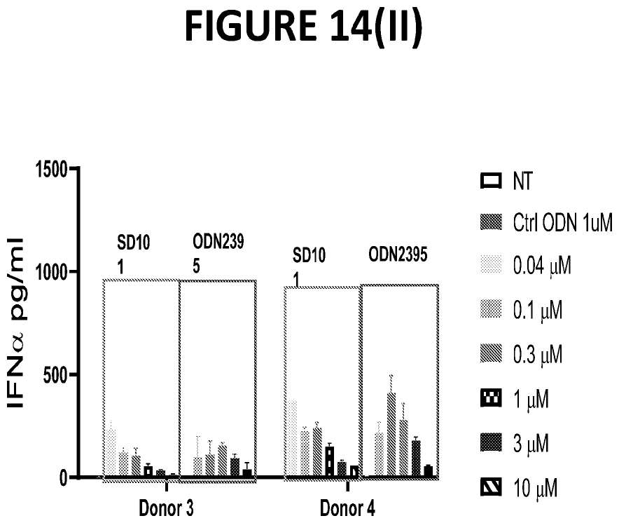

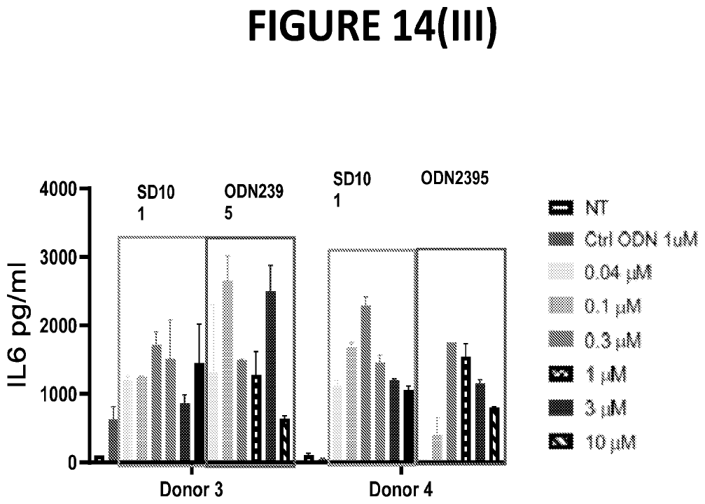

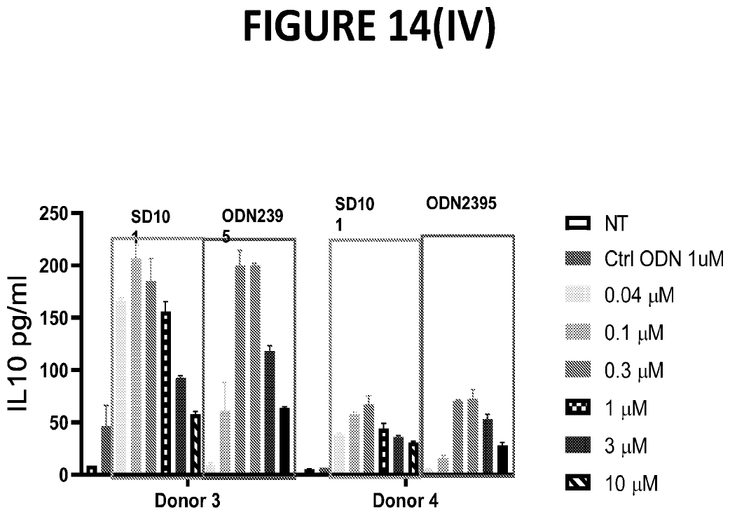

To evaluate the effect of class C TLR9 agonists on huMDSC population (CD11b⁺CD33⁺HLA-DR^{lo}), isolated human PBMCs from healthy donors were treated with increasing concentrations (0.04-10 μM) of class C ODN2395 and SD-101, and sequence control ODN5328 (1 μM), for 48 hours (FIG. 9A). It was found that both SD-101 and ODN2395 decreased the huMDSC population (FIG. 9B), which was quantified via FC analysis. Four donors with three replicates were used, and data represented as mean±SEM (n=12). However, the MDSC-diminishing effect decreased with increased concentrations (3 μM and 10 μM) of SD-101. Further, 0.3 UM dose for both TLR9 agonists seemed to be optimal in decreasing the huMDSC population. The cell supernatants were analyzed by Luminex for IL6, IL10, IL29 and IFNα (FIG. 9C and FIG. 14). Cells treated with SD-101 and ODN2395 are represented with respective boxes. All donors (n=4) responded to the class C TLR9 agonists in a biphasic manner. The Luminex analysis performed on supernatant collected from cells treated (0.04-10 μM) of SD-101 and ODN2395, and sequence control ODN5328 (1 μM), for 48 hours. For donors 1 and 2, supernatants from 10 μM ODN2395 treated samples were unavailable for Luminex analysis. Class C TLR9 agonist-mediated cytokine induction was initiated at 6 hours post treatment. There was a donor-to-donor baseline variability in the cytokine production although they all had similar patterns in the cytokine production by huPBMCs treated with SD-101 or ODN2395. Further, with regard to FIG. 14, human PBMCs were isolated from Donors 3 and 4 and were treated with increasing concentrations (0.04-10 μM) of SD-101 and ODN2395, and control ODN5328 (1 μM), for 48 hours. Supernatants were then analyzed for (i) IL29, (ii) IFNα, (iii) IL6 and (iv) IL10 using a Luminex assay.

TLR9 is Expressed in Human LM Tissue and on the Surface of huMDSCs

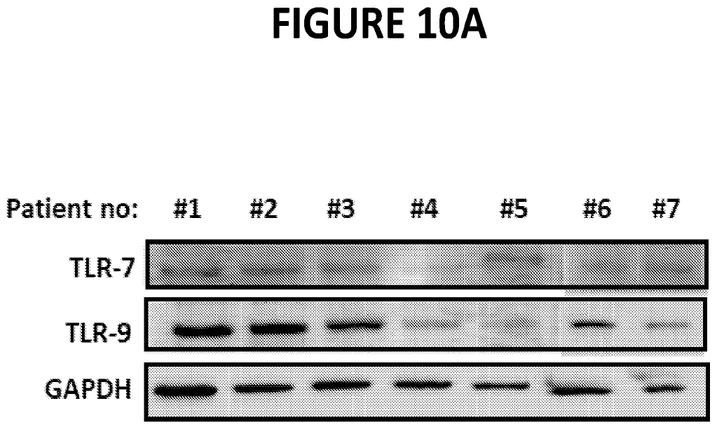

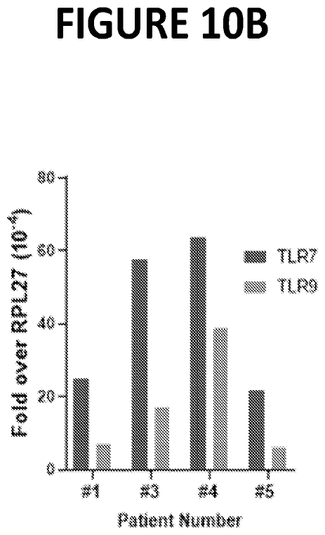

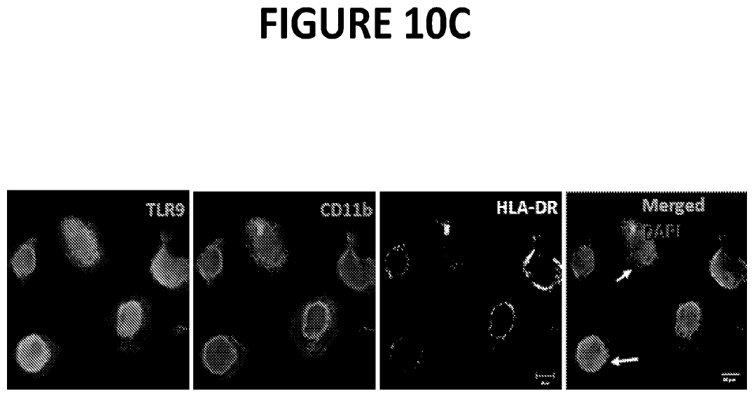

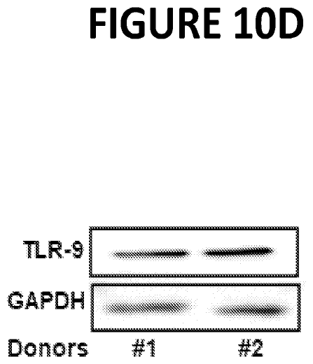

Preclinical murine data demonstrated that class C TLR9 agonist delivered via PV reduced LM burden, possibly by altering the TME and enabling anti-tumor immunity. Functional data confirmed that ODN2395 and SD-101 mediated increase in pro-inflammatory cytokine is TLR9 dependent and decreased MDSC cell population in huPBMCs. We confirmed the expression of TLR9 and related endosomal protein TLR7 in the LM samples at protein and transcript levels on the tissues obtained from seven different cancer patients (FIGS. 10A and 10B). FIG. 10A illustrates protein lysates obtained from LM patient biospecimens that were evaluated for TLR7 and TLR9 by WB. GAPDH was used as housekeeping protein control. WB was performed on two different runs (#1 to #5 on one run, and #6 and #7 were performed in a different run). FIG. 10B illustrates total RNA isolated from the same biospecimen and corresponding TLR9 expression, as quantified by qRT-PCR. RPL-27 gene was used as a housekeeping control. Thus, the presence of TLR9 in human LM samples demonstrates the potential for regional delivery of a TLR9 agonist such as SD-101 to recapitulate preclinical murine efficacy when administered in a clinical setting.

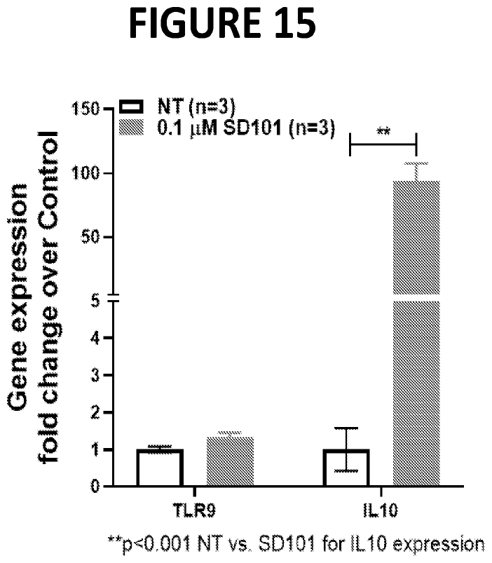

TLR9 is predominantly expressed in the endosomal compartment. However, TLR9 is also expressed on the cell surface of splenic DCs, rat peritoneal mast cells, and in certain experimental settings. In this regard, IL6 (20 ng/ml)+GMCSF (20 ng/ml) stimulated PBMCs grown in chamber slides were fixed and stained with TLR9, CD11b and HLA-DR antibodies and DAPI used for nuclear staining. Using IF, it was confirmed that huMDSCs (CD11b⁺CD33⁺HLA-DR^{lo/-}) express TLR9 on their surface (FIG. 10C). The data is representative of three different experiments using PBMCs from three different donors. WB data on lysates obtained from IL6 (20 ng/ml)+GMCSF (20 ng/ml) treated huPBMCs further confirmed the expression of TLR9 in the MDSC-enriched cells (FIG. 10D, in which GAPDH was used as a control (the data is representative of two out of four donors)). Further, qRT-PCR data of CD11b⁺Gr1⁺ magnetically beaded MDSCs from mouse LM also confirmed the expression of TLR9 transcripts (FIG. 15), and SD-101 did not alter the expression of TLR9 transcripts. In this regard, MDSCs were isolated from mouse LM using CD11b⁺Gr1⁺ negative selection method. Cells were then treated with SD-101 for 24 hours. Isolated RNA was then analyzed for TLR9 and IL10 by qRT-PCR. GAPDH was used as housekeeping gene control. MDSCs were isolated from 3 independent animals and mean±SEM was plotted in the graph.

Class C TLR9 Agonists Inhibit the Differentiation of huMDSCs from huPBMCs

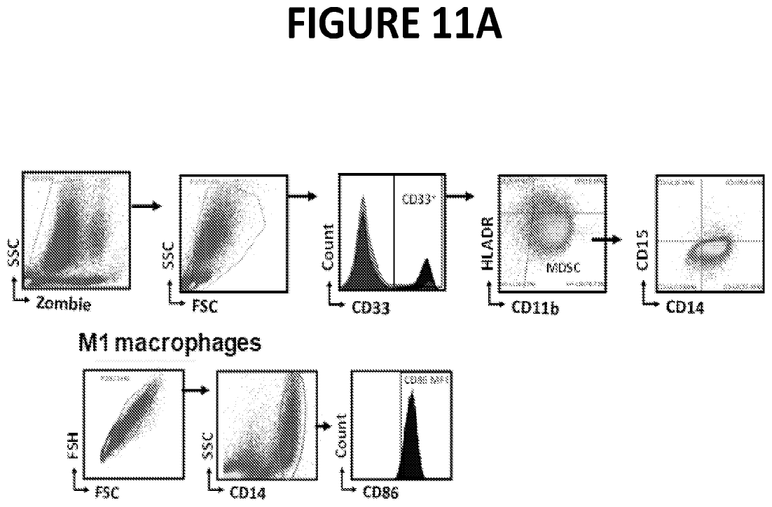

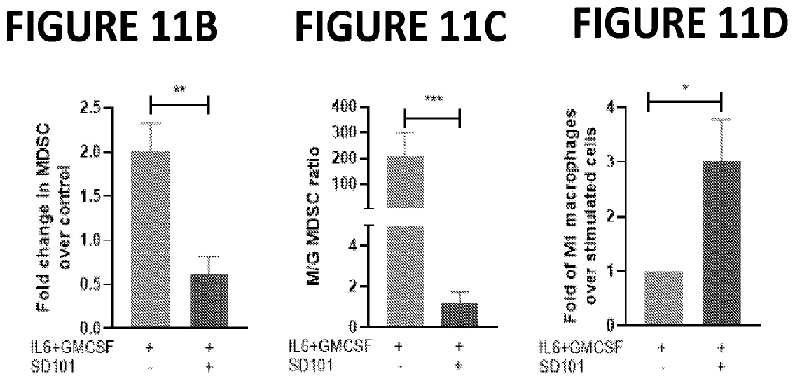

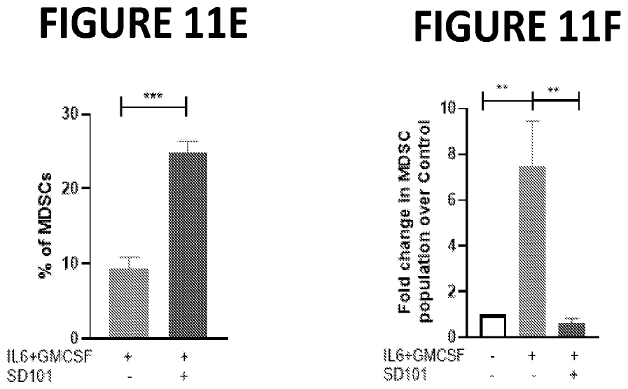

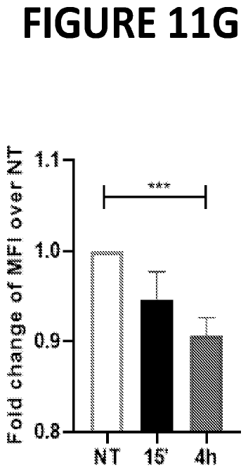

To investigate the effect of SD-101 on the differentiation of huMDSCs from PBMCs, huPBMCs were stimulated with IL6 (20 ng/ml)+GMCSF (20 ng/ml), and treated with SD-101 for seven days to induce the cytokine and growth factor induced MDSC transformation shown in and identified in huMDSC, as shown in FIG. 11A (gating strategy to identify huMDSCs, its subtypes M- and G-MDSCs, and M1 macrophages). In this regard, PBMCs were treated with IL6 (20 ng/ml)+GMCSF (20 ng/ml) for 7 days in the presence or absence of 0.3 μM SD-101. SD-101 treatment significantly reduced the huMDSC population (FIG. 11B, which shows the percentage of MDSCs (CD11b⁺CD33⁺HLA-DR^{lo/-}) after the cells were treated with SD-101 on D0, D2 and D7). Furthermore, similar to the murine LM model, SD-101 preferentially reduced the M-MDSC subset (FIG. 11C, which shows the ratio of M-/G-MDSCs (M-MDSCs: CD11b⁺CD14⁺CD15⁻HLA-DR^{lo/-}, G-MDSCs:CD11b⁺CD14⁻CD15⁺HLA-DR^{lo/-})) and significantly increased (3-fold) M1 macrophage polarization (FIG. 11D, which shows the macrophage population (CD14⁺CD86⁺)). Further SD-101 induced (9.28 vs. 24.81; p<0.001) MDSC apoptosis as measured by Annexin V positive cells (FIG. 11E). Further, a single treatment with SD-101 was sufficient to inhibit huMDSC differentiation (FIG. 11F, which shows the MDSC population after PBMCs were treated with SD-101 once on DO with SD-101 (0.3 μM) for 48 hours).

It was hypothesized that SD-101 would inhibit the STAT3 phosphorylation of MDSCs, thereby inhibiting their expansion. HuMDSCs was generated by treating huPBMCs with IL6+GMCSF for six days. On day 6, enriched MDSCs were treated with SD-101 (0.3 μM) for 15 minutes or 4 hours. FC analysis was performed to quantify pSTAT3 MFI in the MDSC gated cells and reported as fold change in the MFI of pSTAT3 positive cells. All the experiments were performed at least three times, and mean±SEM was plotted in the graph. FC analysis showed significantly reduced phosphorylation of STAT3 (p<0.05) in cells treated with SD-101 for 4 hours as compared to NT group (FIG. 11G).

### Regional Administration of a TLR9 Agonist Potentiated the Responsiveness of Anti-PD-1 Therapy

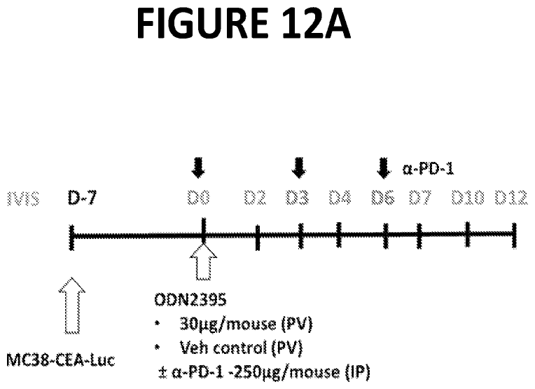

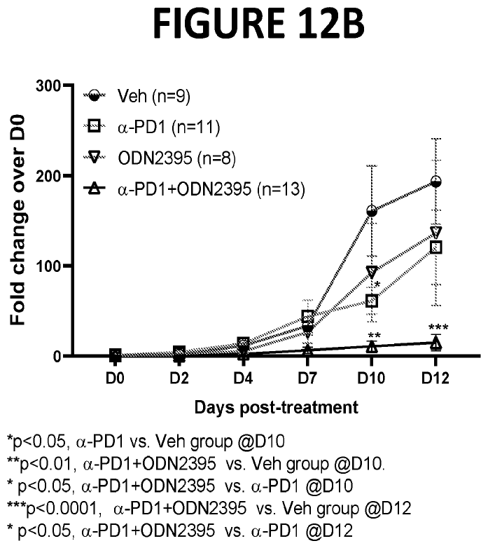

The impact of single-agent regional class C TLR9 agonist treatment on systemic CPI responsiveness was also tested in order to model an ongoing phase 1/1b study for UM LM. Mice with established LM were treated with ODN2395 (30 μg/mouse) via PV, with or without systemic anti-PD1 antibody (α-PD1: 250 μg/mouse) via IP as shown in FIG. 12A. Specifically, eight- to twelve-week-old male C₅₇/BL6 mice were challenged using intra-splenic injection model with MC38-CEA-Luc cells for one week. Bioluminescence was measured by IVIS and mice were randomized on DO prior to treatment with 30 μg/mouse ODN2395 via PV with or without 250 μg/mouse of anti-PD1 antibody via IP on D0, D3 and D6. PBS-treated mice via PV were used as control. Regional intravascular class C TLR9 stimulation significantly enhanced the ability of systemic CPI therapy to control LM burden compared to vehicle treated group (fold change over DO: 14.99 vs. 193.5 @D12; p<0.001) as well as single-agent α-PD1 (fold change over D0: 14.99 vs. 120.4 @D12; p<0.05) and ODN22395 (fold change over D0: 14.99 vs. 136.5 @D12; p=0.08) (FIG. 12B, which shows tumor growth as monitored by IVIS imaging on D2, D4, D7, D10 and D12). In this regard, Fold change of the tumor burden was calculated based on D0 baseline bioluminescence. Data was analyzed by the multiple t test. Combinatorial treatment initiated this antitumor effect beginning at D4 (fold change over D0: 2.49 vs. 11.45; p<0.05).

### DISCUSSION

Regional intravascular delivery of a class C TLR9 agonist in a murine model of PEDD™ enhanced control of LM, favorably reprogramed liver myeloid populations, and enabled systemic CPI. The effect of class C TLR9 agonist on MDSCs was confirmed in both the murine liver and with human PBMCs in vitro. While MDSCs are important drivers of intrahepatic immunosuppression and CPI failure, liver immune dysfunction is likely the result of a complex network of multiple factors. Class C TLR9 agonists can stimulate both adaptive and innate immunity through multiple cell types to potentiate antitumor immune responses.

The liver is a unique organ which is intrinsically immunosuppressive due to the presence of suppressive cells such as MDSC and Tregs, in addition to cytokines secreted by these cells such as IL 10 and TGFβ. The intrahepatic space contains an abundance of MDSCs in the presence of tumor which are key drivers of the immunosuppressive TME. The extent of MDSC expansion is dependent on the tumor burden and the extent of the disease. In this regard, MDSCs have the ability to adapt to organ-specific environmental cues, and when exposed to the intrahepatic space, adopt a specific molecular program while skewing toward the M-MDSC subtype. Here, a decrease in total liver MDSCs and a relative reduction in M-MDSCs was observed in mice with LM treated with a class C TLR9 agonist via PV. The suppressive nature of the liver itself and TMEs make regional intravascular infusion of a TLR9 agonist attractive such that immune cells throughout the organ and within all intrahepatic tumors may be treated.

The present study demonstrated monotherapy activity with a class C TLR9 agonist when delivered regionally, and that a more profound control of LM was achieved when combined with systemic CPI. It was demonstrated that regional TLR9 agonist infusions addressed a critical driver of intrahepatic immunosuppression by reducing liver MDSCs in association with STAT3 deactivation, in addition to supporting favorable MDSC and macrophage polarization. The present study also demonstrated that, following regional class C TLR9 agonist infusion, STAT3 activation induced liver MDSC apoptosis.

M1 macrophages can be activated by TLR agonists and IFNγ and elicit inflammatory responses and antitumor immunity. In contrast, M2 macrophages promote immunosuppression and pro-tumorigenic activities. Plasticity of macrophages is dependent on multiple signals in the TME and the polarization state at any given point in time is not fixed. The present study has also demonstrated that class C TLR9 agonists can drive immunogenic polarization of macrophages through increased M1/M2 ratios, supporting a more pro-inflammatory and anti-tumorigenic TME.

Activation of both the NFκB and STAT3 pathways enhance the expansion and accumulation of MDSCs in the tumor. In this study, following class C TLR9 agonist treatment via PV, pNFκB and IL6 signaling with reduced pSTAT3 activation within LM was observed. STAT3 is considered a protooncogene and is persistently phosphorylated in many cancers including hepatocellular carcinomas. STAT3 has a role in tumor immunity by promoting pro-oncogenic inflammatory pathways, including nuclear factor-κB (NF-κB) and interleukin-6 (IL-6)-GP130-Janus kinase (JAK) pathways. Further, there is a biphasic regulation of NFκB-dependent signaling for SD-101 and ODN2395. Further, it was also demonstrated that SD-101 induced IFNα and IL10 in huPBMC in a “bell shaped curve” dose response.

Activation of transcription factor NFκB could initiate anti- or pro-apoptotic signaling depending on the cell type where it is expressed. For example, DNA-damaging agents such as daunorubicin and serum withdrawal from HEK293 cells or Sindbis-Virus-induced apoptosis in a carcinoma cell line all cause NFκB activation-induced apoptosis. In this study, it was found that SD-101 induced apoptosis in the huMDSC population.

This study has shown that stimulating the TME with a regionally delivered class C TLR9 agonist can enhance the anti-PD-1 antitumor effect for LM up to day 12 post first treatment.

In conclusion, the study demonstrated that class C TLR9 agonists can alter the TME in LM, by eradicating MDSCs and favorably polarizing liver myeloid cells to blunt the impact of the highly immunosuppressive intrahepatic space on systemic CPI.

In some embodiments, the present invention relates to the use of CPI in the manufacture of a medicament for treating a solid tumor in the liver, such as a tumor that is the metastasis of a colorectal cancer, said method comprising administering CPI to a patient in need thereof, wherein CPI is administered through a device by HAI to such solid tumor in the liver.

In some embodiments, the present invention relates to the use of CPI in the manufacture of a medicament for treating pancreatic cancer, said method comprising administering CPI to a patient in need thereof, wherein CPI is administered through a device by PRVI to a solid tumor in the pancreas.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method of treating metastases of colorectal cancer to a liver, said method comprising administering a therapeutically effective amount of checkpoint inhibitor (CPI) to a subject in need thereof.

2. The method of claim 1, wherein the CPI is administered through a device by hepatic arterial infusion (HAI).

3. The method of claim 1, wherein the CPI comprises a Programmed Death 1 receptor (PD-1) antagonist.

4. The method of claim 3, wherein the PD-1 antagonist is at least one of nivolumab, pembrolizumab, and cemiplimab.

5. The method of claim 1, wherein the CPI comprises a Programmed Cell Death 1 Ligand 1 (PD-L1) antagonist.

6. The method of claim 5, wherein the PD-L1 antagonist is at least one of atezolizumab, avelumab, and durvalumab.

7. The method of claim 1, wherein the CPI is administered through a catheter device.

8. The method of claim 7, wherein the catheter device comprises a one-way valve that responds dynamically to local pressure and/or flow changes.

9. The method of claim 8, wherein the CPI is administered through the catheter device via pressure-enabled drug delivery.

10. The method of claim 1, wherein the therapeutically effective amount of the CPI is selected from the range of 0.01-10 mg/kg.

11. A method of treating pancreatic cancer, said method comprising administering a therapeutically effective amount of checkpoint inhibitor (CPI) to a subject in need thereof.

12. The method of claim 11, wherein the CPI is administered through a device by pancreatic retrograde venous infusion (PRVI).

13. The method of claim 11, wherein the CPI comprises a Programmed Death 1 receptor (PD-1) antagonist.

14. The method of claim 13, wherein the PD-1 antagonist is at least one of nivolumab, pembrolizumab, and cemiplimab.

15. The method of claim 11, wherein the CPI comprises a Programmed Cell Death 1 Ligand 1 (PD-L1) antagonist.

16. The method of claim 15, wherein the PD-L1 antagonist is at least one of atezolizumab, avelumab, and durvalumab.

17. The method of claim 11, wherein the CPI is administered through a catheter device.

18. The method of claim 17, wherein the CPI is administered through the catheter device via pressure-enabled drug delivery.

19. The method of claim 11, wherein the therapeutically effective amount of the CPI is selected from the range of 0.01-10 mg/kg.


SEQUENCE LISTING (1 sequence)

SEQ ID NO: 1 - DNA, 30 bp, Artificial Sequence
  Description of Artificial Sequence Synthetic oligonucleotide
tcgaacgttcgaacgttcgaacgttcgaat